(19) United States
(12) Patent Application Publication
(10) Pub. No.: US 2019/0125897 A1
(43) Pub. Date: May 2, 2019

(54) ANTIMICROBIAL AGENTS AGAINST SALMONELLA BACTERIA

(71) Applicant: LYSANDO AG, Triesenberg (LI)

(72) Inventors: Martin GRIESSL, Hohenschambach (DE); Manfred BIEBL, Obertraubling (DE); Eva SCHIRMEIER, Regensburg (DE)

(21) Appl. No.: 16/097,142

(22) PCT Filed: Apr. 28, 2017
(86) PCT No.: PCT/EP2017/060264

§ 371 Date: Oct. 26, 2018

(30) Foreign Application Priority Data
Apr. 28, 2016 (EP) 16167543.4

(51) Int. Cl. A61K 48/00; C07K 14/46; C12N 15/62; A61P 31/04

(52) U.S. Cl. CPC A61K 48/005; A61P 31/04; C12N 15/62; C07K 14/46

(57) ABSTRACT

The present invention relates to the field of antimicrobial agents active against Salmonella bacteria. In particular, the present invention relates to polypeptides comprising a first and a second amino acid sequence, wherein the first amino acid sequence is an endolysin, and wherein the second amino acid sequence is an antimicrobial peptide, cationic peptide, hydrophobic peptide, amphipathic peptide or sushi peptide, and wherein the polypeptide comprises at least one sequence selected from the group consisting of SEQ ID NO:1, SEQ ID NO:3, SEQ ID NO:4 and derivatives thereof. In addition, the present invention relates to nucleic acids encoding such polypeptides, vectors comprising such nucleic acids, and corresponding host cells. Finally, the present invention relates to applications of the inventive polypeptides, nucleic acids, vectors, and/or host cells, in particular in the pharmaceutical field and in the field of food and feed.

The present invention relates to the field of antimicrobial agents active against Salmonella bacteria. In particular, the present invention relates to polypeptides comprising a first and a second amino acid sequence, wherein the first amino acid sequence is an endolysin, and wherein the second amino acid sequence is an antimicrobial peptide, cationic peptide, hydrophobic peptide, amphipathic peptide or sushi peptide, and wherein the polypeptide comprises at least one sequence selected from the group consisting of SEQ ID NO: 1, SEQ ID NO:3, SEQ ID NO:4 and derivatives thereof. In addition, the present invention relates to nucleic acids encoding such polypeptides, vectors comprising such nucleic acids, and corresponding host cells. Finally, the present invention relates to applications of the inventive polypeptides, nucleic acids, vectors, and/or host cells, in particular in the pharmaceutical field and in the field of food and feed.

Bacterial pathogens represent a significant threat for human health. Although various types of agents having bactericidal or bacteriostatic activity are known in the art (e.g. antibiotics), microbial resistance to these, in particular to antibiotics, is steadily increasing. One of the pathogens representing a health concern are bacteria of the genus Salmonella. A large number of infections caused by Salmonella bacteria are due to ingestion of contaminated food. Since increasing resistance renders the use of antibiotics in, e.g., feed additives problematic, there is a constant demand for new antimicrobial agents to control the number of Salmonella bacteria, e.g. in food and feed.

Bacteria of the genus Salmonella are Gram-negative bacteria. Gram-negative bacteria possess an outer membrane, with its characteristic asymmetric bilayer as a hallmark. The outer membrane bilayer consists of an inner monolayer containing phospholipids (primarily phosphatidyl ethanolamine) and an outer monolayer that is mainly composed of a single glycolipid, lipopolysaccharide (LPS). There is an immense diversity of LPS structures in the bacterial kingdom and the LPS structure may be modified in response to prevailing environmental conditions. The stability of the LPS layer and interaction between different LPS molecules is mainly achieved by the electrostatic interaction of divalent ions (Mg²⁺, Ca²⁺) with the anionic components of the LPS molecule (phosphate groups in the lipid A and the inner core and carboxyl groups of KDO). Furthermore, the dense and ordered packing of the hydrophobic moiety of lipid A, favoured by the absence of unsaturated fatty acids, forms a rigid structure with high viscosity. This makes it less permeable for lipophilic molecules and confers additional stability to the outer membrane (OM).

The present invention makes use of endolysins. Endolysins are peptidoglycan hydrolases typically encoded by bacteriophages (or bacterial viruses). They are synthesized during late gene expression in the lytic cycle of phage multiplication and mediate the release of progeny virions from infected cells through degradation of the bacterial peptidoglycan. They are either ß(1,4)-glycosyl hydrolases (lysozymes), transglycosylases, amidases or endopeptidases. Antimicrobial application of endolysins was already suggested in 1991 by Gasson (GB2243611). Although the killing capacity of endolysins has been known for a long time, the use of these enzymes as antibacterials was ignored due to the success and dominance of antibiotics. Only after the appearance of multiple antibiotic resistant bacteria this simple concept of combating human pathogens with endolysins received interest. A compelling need to develop totally new classes of antibacterial agents emerged and endolysins used as ‘enzybiotics’—a hybrid term of ‘enzymes’ and ‘antibiotics’—perfectly met this need. In 2001, Fischetti and coworkers demonstrated for the first time the therapeutic potential of bacteriophage Cl endolysin towards group A streptococci (Nelson et al., 2001, Proc. Natl. Acad. Sci. U.S.A 98:4107-4112; herewith incorporated by reference). Since then many publications have established endolysins as an attractive and complementary alternative to control bacterial infections of Gram-positive bacteria. Subsequently different endolysins against other Gram-positive pathogens such as Streptococcus pneumoniae (Loeffler et al., 2001, Science 294:2170-2172), Bacillus anthracis (Schuch et al., 2002; Nature 418:884-889), S. agalactiae (Cheng et al., 2005; Antimicrob Agents Chemother. 2005 January; 49(1):111-117) and Staphylococcus aureus (Rashel et al, 2007; J Infect Dis. 2007 Oct. 15; 196(8):1237-1247) have proven their efficacy as enzybiotics (all references incorporated herewith by reference). Nowadays, the most important challenge of endolysin therapy lies in the insensitivity of Gram-negative bacteria towards the exogenous action of endolysins, since the outer membrane shields the access of endolysins from the peptidoglycan. This currently prevents the expansion of the range of effective endolysins to important Gram-negative pathogens, such as bacteria of the genus Salmonella.

In the art combinations of endolysins with further amino acid sequence stretches have been described to create new antimicrobial agents. WO 2015/071436 (herewith incorporated by reference) discloses fusions of peptides with derivatives of endolysin KZ144, which show activity towards E. coli, P. aeruginosa, and C. jejuni. Briers et al. (MBio; 2014; 5(4):e01379-14; herewith incorporated by reference) report that fusion proteins of the endolysins OBPgp279 and PVP-SE1gp146 with certain peptides yield antimicrobial agents with moderate activity against Salmonella Typhimurium LT 2 bacteria.

Nonetheless, there is still a constant need for new antibacterial agents active against Gram-negative bacteria. In particular, there is a need for antibacterial agents active against bacteria of the Genus Salmonella. Preferably, said agents are active against a diverse set of Salmonella strains, exhibit an increased activity and/or are sufficiently (heat) stable for technical applications.

This object is solved by the subject matter defined in the claims and set forth below.

The term “polypeptide” as used herein refers in particular to a polymer of amino acids linked by peptide bonds in a specific sequence. The amino acid residues of a polypeptide may be modified by e.g. covalent attachments of various groups such as carbohydrates and phosphate.

Other substances may be more loosely associated with the polypeptide, such as heme or lipid, giving rise to conjugated polypeptides which are also comprised by the term “polypeptide” as used herein. The term as used herein is intended to encompass also proteins. Thus, the term “polypeptide” also encompasses for example complexes of two or more amino acid polymer chains. The term “polypeptide” does encompass embodiments of polypeptides which exhibit optionally modifications typically used in the art, e.g. biotinylation, acetylation, pegylation, chemical changes of the amino-, SH- or carboxyl-groups (e.g. protecting groups) etc. As will become apparent from the description below, the polypeptide according to the present invention may also be a fusion protein, i.e. linkage of at least two amino acid sequences which do not occur in this combination in nature. The term “polypeptide”, as used herein, is not limited to a specific length of the amino acid polymer chain, but typically the polypeptide will exhibit a length of more than about 50 amino acids, more than about 100 amino acids or even more than about 150 amino acids. Usually, but not necessarily, a typical polypeptide of the present invention will not exceed about 750 amino acids in length.

The term “endolysin” as used herein refers to a bacteriophage-derived enzyme which is suitable to hydrolyse bacterial cell walls. Endolysins comprise at least one “enzymatically active domain” (EAD) having at least one of the following activities: endopeptidase, chitinase, T4 like muraminidase, lambda like muraminidase, N-acetyl-muramoyl-L-alanine-amidase (amidase), muramoyl-L-alanine-amidase, muramidase, lytic transglycosylase (C), lytic transglycosylase (M), N-acetyl-muramidase (lysozyme), N-acetyl-glucosaminidase or transglycosylases as e.g. KZ144. In addition, the endolysins may contain also regions which are enzymatically inactive, and bind to the cell wall of the host bacteria, the so-called CBDs (cell wall binding domains). The term “endolysin” also encompasses enzymes which comprise modifications and/or alterations vis-a-vis naturally occurring endolysins. Such alterations and/or modifications may comprise mutations such as deletions, insertions and additions, substitutions or combinations thereof and/or chemical changes of the amino acid residues. Particularly preferred chemical changes are biotinylation, acetylation, pegylation, chemical changes of the amino-, SH- or carboxyl-groups. Said endolysins exhibit on a general level the lytic activity of the respective wild-type endolysin. However, said activity can be the same, higher or lower as the activity of the respective wild-type endolysin. Said activity can be for example at least about 50, 60, 70, 80, 90, 100, 110, 120, 130, 140, 150, 160, 170, 180, 190 or at least about 200% of the activity of the respective wild-type endolysin or even more. The activity can be measured by assays well known in the art by a person skilled in the art as e.g. the plate lysis assay or the liquid lysis assay which are e.g. described in Briers et al. (J. Biochem. Biophys Methods; 2007; 70: 531-533) or Donovan et al. (J. FEMS Microbiol Lett. 2006 December; 265(1) and similar publications.

The term “fragment” as used herein refers to an amino acid sequence which is N-terminally, C-terminally, and/or on both termini truncated with respect to the respective reference sequence, for example a given SEQ ID NO. Thus, a fragment of an amino acid sequence as used herein is an amino acid sequence which is at least one amino acid shorter than the respective reference sequence. A fragment of an amino acid sequence as used herein is preferably an amino acid sequence which is at most 20, more preferably at most 19, more preferably at most 18, more preferably at most 17, more preferably at most 16, more preferably at most 15, more preferably at most 14, more preferably at most 13, more preferably at most 12, more preferably at most 11, more preferably at most 10, more preferably at most 9, more preferably at most 8, more preferably at most 7, more preferably at most 6, more preferably at most 5, more preferably at most 4, more preferably at most 3, more preferably at most 2, most preferably 1 amino acid residue shorter than the respective reference amino acid sequence. The fragment may for example exhibit vis-à-vis the reference sequence a truncation of 1, 2, 3, 4, 5, 6, 7, 8, 9, 10, 11, 12, 13, 14, 15, 16, 17, 18, 19 or 20 amino acids at the N-terminus, the C-terminus or both. It is understood that a polypeptide comprising a fragment of a given amino acid sequence does not comprise the full length of said reference amino acid sequence.

The term “derivative”, as used herein, refers to an amino acid sequence which exhibits, in comparison to the respective reference sequence, one or more additions, deletions, insertions, and/or substitutions and combinations thereof. This includes for example combinations of deletions/insertions, insertions/deletions, deletions/additions, additions/deletions, insertion/additions, additions/insertions etc. A person skilled in the art will however understand that the presence of an amino acid residue at a certain position of the derivative sequence which is different from the one that is present at the respective same position in the reference sequence is not a combination of, for example, a deletion and a subsequent insertion at the same position but is a substitution as defined herein. Rather, if reference is made herein to combinations of one or more of additions, deletions, insertions, and substitutions, then combination of changes at distinct positions in the sequence are intended, e.g. an addition at the N-terminus and an intrasequential deletion. Such derived sequence will exhibit a certain level of sequence identity with the respective reference sequence, for example a given SEQ ID NO, which is preferably at least 60%, such as at least 75%, at least 80%, at least 85%, at least 90%, at least 95%, at least 96%, at least 97%, at least 98% or at least 99%. Preferred derivatives are fragments of the parent molecule, for example a given SEQ ID NO, retaining the activity of the parent molecule, i.e. exhibiting on a general level same activity as the respective parent molecule. However, said activity can be the same, higher or lower as the respective parent molecule. Also preferred derivatives are those resulting from conservative amino acid substitutions within the parent sequence, for example a given SEQ ID NO, again retaining the activity of the parent molecule on a general level.

As used herein, the term “% sequence identity”, has to be understood as follows: Two sequences to be compared are aligned to give a maximum correlation between the sequences. This may include inserting “gaps” in either one or both sequences, to enhance the degree of alignment. A % identity may then be determined over the whole length of each of the sequences being compared (so-called global alignment), that is particularly suitable for sequences of the same or similar length, or over shorter, defined lengths (so-called local alignment), that is more suitable for sequences of unequal length. In the above context, an amino acid sequence having a “sequence identity” of at least, for example, 95% to a query amino acid sequence, is intended to mean that the sequence of the subject amino acid sequence is identical to the query sequence except that the subject amino acid sequence may include up to five amino acid alterations per each 100 amino acids of the query amino acid sequence. In other words, to obtain an amino acid sequence having a sequence of at least 95% identity to a query amino acid sequence, up to 5% (5 of 100) of the amino acid residues in the subject sequence may be inserted or substituted with another amino acid or deleted. Methods for comparing the identity and homology of two or more sequences are well known in the art. The percentage to which two sequences are identical can for example be determined by using a mathematical algorithm. A preferred, but not limiting, example of a mathematical algorithm which can be used is the algorithm of Karlin et al. (1993), PNAS USA, 90:5873-5877. Such an algorithm is integrated in the BLAST family of programs, e.g. BLAST or NBLAST program (see also Altschul et al., 1990, J. Mol. Biol. 215, 403-410 or Altschul et al. (1997), Nucleic Acids Res, 25:3389-3402), accessible through the home page of the NCBI at world wide web site ncbi.nlm.nih.gov) and FASTA (Pearson (1990), Methods Enzymol. 83, 63-98; Pearson and Lipman (1988), Proc. Natl. Acad. Sci. U. S. A 85, 2444-2448.). Sequences which are identical to other sequences to a certain extent can be identified by these programmes. Furthermore, programs available in the Wisconsin Sequence Analysis Package, version 9.1 (Devereux et al, 1984, Nucleic Acids Res., 387-395), for example the programs BESTFIT and GAP, may be used to determine the % identity between two polypeptide sequences. BESTFIT uses the “local homology” algorithm of (Smith and Waterman (1981), J. Mol. Biol. 147, 195-197.) and finds the best single region of similarity between two sequences. If herein reference is made to an amino acid sequence sharing a particular extent of sequence identity to a reference sequence, then said difference in sequence is preferably due to conservative amino acid substitutions. Preferably, such sequence retains the activity of the reference sequence, e.g. albeit maybe at a slower rate. In addition, if reference is made herein to a sequence sharing “at least” at certain percentage of sequence identity, then 100% sequence identity are preferably not encompassed.

“Conservative amino acid substitutions”, as used herein, may occur within a group of amino acids which have sufficiently similar physicochemical properties, so that a substitution between members of the group will preserve the biological activity of the molecule (see e.g. Grantham, R. (1974), Science 185, 862-864). Particularly, conservative amino acid substitutions are preferably substitutions in which the amino acids originate from the same class of amino acids (e.g. basic amino acids, acidic amino acids, polar amino acids, amino acids with aliphatic side chains, amino acids with positively or negatively charged side chains, amino acids with aromatic groups in the side chains, amino acids the side chains of which can enter into hydrogen bridges, e.g. side chains which have a hydroxyl function, etc.). Conservative substitutions are in the present case for example substituting a basic amino acid residue (Lys, Arg, His) for another basic amino acid residue (Lys, Arg, His), substituting an aliphatic amino acid residue (Gly, Ala, Val, Leu, lie) for another aliphatic amino acid residue, substituting an aromatic amino acid residue (Phe, Tyr, Trp) for another aromatic amino acid residue, substituting threonine by serine or leucine by isoleucine. Further conservative amino acid exchanges will be known to the person skilled in the art.

The term “deletion” as used herein refers preferably to the absence of 1, 2, 3, 4, 5 (or even more than 5) continuous amino acid residues in the derivative sequence in comparison to the respective reference sequence, either intrasequentially or at the N- or C-terminus. A derivative of the present invention may exhibit one, two or more of such deletions.

The term “insertion” as used herein refers preferably to the additional intrasequential presence of 1, 2, 3, 4, 5 (or even more than 5) continuous amino acid residues in the derivative sequence in comparison to the respective reference sequence. A derivative of the present invention may exhibit one, two or more of such insertions.

The term “addition” as used herein refers preferably to the additional presence of 1, 2, 3, 4, 5 (or even more than 5) continuous amino acid residues at the N- and/or C-terminus of the derivative sequence in comparison to the respective reference sequence.

The term “substitution” as used herein refers to the presence of an amino acid residue at a certain position of the derivative sequence which is different from the amino acid residue which is present or absent at the corresponding position in the reference sequence. A derivative of the present invention may exhibit 1, 2, 3, 4, 5, 6, 7, 8, 9, 10, 11, 12, 13, 14, 15, 16, 17, 18, 19, 20, or more of such substitutions. As mentioned above, preferably such substitutions are conservative substitutions.

The term “cell wall” as used herein refers to all components that form the outer cell enclosure of Gram-negative bacteria and thus guarantee their integrity. In particular, the term “cell wall” as used herein refers to peptidoglycan, the outer membrane of the Gram-negative bacteria with the lipopolysaccharide, the bacterial cell membrane, but also to additional layers deposited on the peptidoglycan as e.g. capsules, outer protein layers or slimes.

The term “second amino acid sequence”, as used herein refers an amino acid subsequence within the amino acid sequence of the polypeptide of the invention. Said sequence may be the sequence of a cationic peptide, a polycationic peptide, an amphipathic peptide, a hydrophobic peptide, a sushi peptide and/or an antimicrobial peptide. The term does not refer to conventional tags like His-tags, such as His5-tags, His6-tags, His7-tags, His8-tags, His9-tags, His10-tags, His11-tags, His12-tags, His16-tags and His20-tags, Strep-tags, Avi-tags, Myc-tags, Gst-tags, JS-tags, cystein-tags, FLAG-tags or other tags known in the art, thioredoxin or maltose binding proteins (MBP). Preferably, the second amino acid sequence has as a length of at least about 6 to at most about 50, preferably at most about 39 amino acid residues.

The terms “first amino acid sequence” and “second amino acid sequence”, as used herein, do not imply an inherent order of the sequences within the inventive polypeptide, i.e. the second amino acid sequence may be N-terminal of the first amino acid sequence or C-terminal of the first amino acid sequence.

As used herein, the term “cationic peptide” refers preferably to a peptide having positively charged amino acid residues. Preferably a cationic peptide has a pKa-value of 9.0 or greater. Typically, at least four of the amino acid residues of the cationic peptide can be positively charged, for example, lysine or arginine. “Positively charged” refers to the side chains of the amino acid residues which have a net positive charge at about physiological conditions. The term “cationic peptide” as used herein refers also to polycationic peptides, but also includes cationic peptides which comprise for example less than 20%, preferably less than 10% positively charged amino acid residues.

The term “polycationic peptide” as used herein refers preferably to a peptide composed of mostly positively charged amino acid residues, in particular lysine and/or arginine residues. A peptide is composed of mostly positively charged amino acid residues if at least about 20, 30, 40, 50, 60, 70, 75, 80, 85, 90, 95 or about 100% of the amino acid residues are positively charged amino acid residues, in particular lysine and/or arginine residues. The amino acid residues being not positively charged amino acid residues can be neutrally charged amino acid residues and/or negatively charged amino acid residues and/or hydrophobic amino acid residues. Preferably the amino acid residues being not positively charged amino acid residues are neutrally charged amino acid residues, in particular serine and/or glycine.

The term, “antimicrobial peptide” (AMP) as used herein refers preferably to any naturally occurring peptide that has microbicidal and/or microbistatic activity on, for example, bacteria, viruses, fungi, yeasts, mycoplasma and protozoa. Thus, the term “antimicrobial peptide” as used herein refers in particular to any peptide having anti-bacterial, anti-fungal, anti-mycotic, anti-parasitic, anti-protozoal, anti-viral, anti-infectious, anti-infective and/or germicidal, algicidal, amoebicidal, microbicidal, bactericidal, fungicidal, parasiticidal, protozoacidal, protozoicidal properties. Preferred are anti-bacterial peptides. The antimicrobial peptide may be a member of the RNase A super family, a defensin, cathelicidin, granulysin, histatin, psoriasin, dermicidine or hepcidin. The antimicrobial peptide may be naturally occurring in insects, fish, plants, arachnids, vertebrates or mammals. Preferably the antimicrobial peptide may be naturally occurring in radish, silk moth, wolf spider, frog, preferably in Xenopus laevis, Rana frogs, more preferably in Rana catesbeiana, toad, preferably Asian toad Bufo bufo gargarizans, fly, preferably in Drosophila, more preferably in Drosophila melanogaster, in Aedes aegypti, in honey bee, bumblebee, preferably in Bombus pascuorum, flesh fly, preferably in Sarcophaga peregrine, scorpion, horseshoe crab, catfish, preferably in Parasilurus asotus, cow, pig, sheep, porcine, bovine, monkey and human. As used herein, an “antimicrobial peptide” (AMP) may in particular be a peptide which is not a cationic peptide, polycationic peptide, amphipathic peptide, sushi peptide, defensins, and hydrophobic peptide, but nevertheless exhibits antimicrobial activity.

The term “sushi peptide” as used herein refers to complement control proteins (CCP) having short consensus repeats. The sushi module of sushi peptides functions as a protein-protein interaction domain in many different proteins. Peptides containing a Sushi domain have been shown to have antimicrobial activities. Preferably, sushi peptides are naturally occurring peptides.

The term “defensin” as used herein refers to a peptide present within animals, preferably mammals, more preferably humans, wherein the defensin plays a role in the innate host defense system as the destruction of foreign substances such as infectious bacteria and/or infectious viruses and/or fungi. A defensin is a non-antibody microbicidal and/or tumoricidal protein, peptide or polypeptide. Examples for “defensins” are “mammalian defensins,” alpha-defensins, beta-defensins, indolicidin and magainins. The term “defensins” as used herein refers both to an isolated form from animal cells or to a synthetically produced form, and refers also to variants which substantially retain the cytotoxic activities of their parent proteins, but whose sequences have been altered by insertion or deletion of one or more amino acid residues.

The term “amphipathic peptide” as used herein refers to synthetic peptides having both hydrophilic and hydrophobic functional groups. Preferably, the term “amphipathic peptide” as used herein refers to a peptide having a defined arrangement of hydrophilic and hydrophobic groups e.g. amphipathic peptides may be e.g. alpha helical, having predominantly non polar side chains along one side of the helix and polar residues along the rest of its surface.

The term “hydrophobic group” as used herein refers preferably to chemical groups such as amino acid side chains which are substantially water insoluble, but soluble in an oil phase, with the solubility in the oil phase being higher than that in water or in an aqueous phase. In water, amino acid residues having a hydrophobic side chain interact with one another to generate a non-aqueous environment. Examples of amino acid residues with hydrophobic side chains are valine, isoleucine, leucine, methionine, phenylalanine, tryptophan, cysteine, alanine, tyrosine, and proline residues

The term “hydrophobic peptide” as used herein refers to a hydrophobic peptide, which is preferably composed of mostly amino acid residues with hydrophobic groups. Such peptide is preferably composed of mostly hydrophobic amino acid residues, i.e. at least about 20, 30, 40, 50, 60, 70, 75, 80, 85, 90, 95 or at least about 100% of the amino acid residues are hydrophobic amino acid residues. The amino acid residues being not hydrophobic are preferably neutral and preferably not hydrophilic.

As used herein, the term “tag” refers to an amino acid sequence, which is typically in the art fused to or included in another amino acid sequence for a) improving expression of the overall amino acid sequence or polypeptide, b) facilitating purification of the overall amino acid sequence or polypeptide, c) facilitating immobilisation of the overall amino acid sequence or polypeptide, and/or d) facilitating detection of the overall amino acid sequence or polypeptide. Examples for tags are His tags, such as His5-tags, His6-tags, His7-tags, His8-tags, His9-tags, His10-tags, His11-tags, His12-tags, His16-tags and His20-tags, Strep-tags, Avi-tags, Myc-tags, GST-tags, JS-tags, cystein-tags, FLAG-tags, HA-tags, thioredoxin or maltose binding proteins (MBP), CAT, GFP, YFP, etc. The person skilled in the art will know a vast number of tags suitable for different technical applications. The tag may for example make such tagged polypeptide suitable for e.g. antibody binding in different ELISA assay formats or other technical applications.

The term “comprising” as used herein shall not be construed as being limited to the meaning “consisting of” (i.e. excluding the presence of additional other matter). Rather, “comprising” implies that optionally additional matter may be present. The term “comprising” encompasses as particularly envisioned embodiments falling within its scope “consisting of” (i.e. excluding the presence of additional other matter) and “comprising but not consisting of” (i.e. requiring the presence of additional other matter), with the former being more preferred.

The inventors of the present invention have surprisingly found that endolysins according to SEQ ID NO:1 and SEQ ID NO:2, and their derivatives, as well as antimicrobial peptides according to SEQ ID NO:3 and SEQ ID NO:4, and their derivatives, are extremely useful components when designing antimicrobial agents against bacteria of the Genus Salmonella. Such compounds show increased activity, for example against Salmonella Typhimurium bacteria.

Therefore, the present invention relates in a first aspect to a polypeptide comprising a first and a second amino acid sequence, wherein the first amino acid sequence is an endolysin, and wherein the second amino acid sequence is an antimicrobial peptide, amphiphatic peptide, cationic peptide, hydrophobic peptide, sushi peptide or defensin, and wherein the polypeptide comprises at least one sequence selected from the following group of sequences:
i) an amino acid sequence according to SEQ ID NO: 1;
ii) a derivative of SEQ ID NO: 1 exhibiting at least 80% sequence identity with SEQ ID NO: 1, with the proviso that the polypeptide may not comprise the sequence of SEQ ID NO:5 if the polypeptide comprises ii), but none of iii), iv), v) or vi),
iii) an amino acid sequence according to SEQ ID NO:3;
iv) a derivative of SEQ ID NO:3 exhibiting at least 77% sequence identity with SEQ ID NO:3;
v) an amino acid sequence according to SEQ ID NO:4, with the proviso that the polypeptide may in this case not comprise the sequence of SEQ ID NO:6 in parallel; and
vi) a derivative of SEQ ID NO:4 exhibiting at least 80% sequence identity with SEQ ID NO:4.

The amino acid sequence according to SEQ ID NO: 1 and derivatives thereof are examples for the first amino acid sequence, i.e. the endolysin, SEQ ID NO:3, SEQ ID NO:4 and respective derivatives are examples for the second amino acid sequence.

In preferred embodiments of the invention the first amino acid sequence is selected from the group consisting of:
i) an amino acid sequence according to SEQ ID NO: 1;
ii) a derivative of SEQ ID NO: 1 exhibiting at least 80% sequence identity with SEQ ID NO:1,
iii) an amino acid sequence according to SEQ ID NO:2; and
iv) a derivative of SEQ ID NO:2 exhibiting at least 80% sequence identity with SEQ ID NO:2.

Derivatives of SEQ ID NO: 1 exhibit preferably at least 85%, at least 87.5%, at least 90%, at least 91%, at least 92%, at least 93%, at least 94%, at least 95%, at least 96%, at least 97%, at least 98%, at least 99%, or more than 99% sequence identity with SEQ ID NO: 1. An example for a derivative of SEQ ID NO: 1 is the amino acid sequence according to SEQ ID NO:7. In other embodiments, the derivative is not an amino acid sequence according to SEQ ID NO:7. Particularly preferred derivatives exhibit at least 98%, at least 99%, or more than 99% sequence identity with SEQ ID NO: 1. As mentioned before, a polypeptide of the present invention comprising a derivative of SEQ ID NO:1 may not comprise the sequence of SEQ ID NO:5, if the polypeptide of the invention does not comprise in parallel an amino acid sequence according to SEQ ID NO:3, a derivative of SEQ ID NO:3 as defined herein, an amino acid sequence according to SEQ ID NO:4, and/or a derivative of SEQ ID NO:4 as defined herein. This proviso applies for the polypeptides of the invention per se, but not necessarily for other aspects of the present invention, e.g. methods and uses employing such polypeptide, in particular when dealing with Salmonella bacteria. However, said proviso may of course also apply to the other aspects, e.g. the methods and uses of such polypeptide.

Derivatives of SEQ ID NO:2 exhibit preferably at least 85%, at least 87.5%, at least 90%, at least 91%, at least 92%, at least 93%, at least 94%, at least 95%, at least 96%, at least 97%, at least 98%, at least 99%, or more than 99% sequence identity with SEQ ID NO:2. Suitable derivatives of SEQ ID NO:2 according to SEQ ID NO:8 have been described extensively in WO 2015/071436 (incorporated herein by reference).

The derivative of SEQ ID NO:2, e.g. based on the consensus sequence according to SEQ ID NO:8, may deviate from SEQ ID NO:2 in particular at those positions in SEQ ID NO:8, which are known to be non-critical for the enzymatic activity, i.e. X1, X14, X23, X50, X82, X122, X149; X160, X167, X179, X180, X186; X206; X212; X224; X230 and/or X232. Such derivative may exhibit at least one (e.g. 1, 2, 3, 4, 5, 6, 7, 8, 9, 10, 11, 12, 13, 14, 15, 16 or even all 17) of the following: X14 is not C; X23 is not C; X50 is not C; X82 is I; X122 is M; X149 is P; X154 is T, X160 is T; X167 is L; X179 is F; X180 is E; X186 is Y; X206 is N or V, X212 is N; X224 is Q; X230 is Y and/or X232 is T. It is understood that the number indicating the position of the respective amino acid residue indicates the relative position in the sequence corresponding to SEQ ID NO:8, and not to the overall amino acid sequence of the polypeptide according to the present invention, which may be longer.

Preferably, any derivative of SEQ ID NO:2 exhibits a glutamic acid residue at the position corresponding to position 114 in SEQ ID NO:2. As shown in the publication Briers et al. (Molecular Microbiology; 2007; 65(5), 1334-1344), the mutation E115A (position 114 in SEQ ID NO:2) led to a loss in activity of about 70% of the enzyme. Thus, while an inventive polypeptide comprising said mutation will thus not be a loss of function polypeptide and may still serve various technical purposes, it is certainly preferred if such mutation is not present in the sequence stretch corresponding to SEQ ID NO:8 within the inventive polypeptide.

In a particular preferred embodiment according to the present invention the derivative of SEQ ID NO:2 comprises the sequence of SEQ ID NO:8.

The authors of WO 2015/071436 have found out that three cysteine residues in the amino acid sequence of SEQ ID NO:2 (KZ144 endolysin sequence) are not essential for the enzymatic activity. Thus, in the sequence corresponding to SEQ ID NO:8 (consensus sequence of selected KZ144 derivatives), in some embodiments X14 is not C, X23 is not C, or X50 is not C. Combinations are possible, e.g. X14 and X23 are not C, X14 and X50 are not C, or X23 and X50 are not C. Likewise, it is also possible that neither X14 nor X23 nor X50 are C. In principle said amino acid residues can be deleted or substituted by any other amino acid. Examples for such other amino acids are S, R and N. Thus, X14 may for example be S, N, or R; more preferably S or R; most preferably R; X23 may for example be S, N, or R, more preferably S; and X50 may for example be S, N, or R, more preferably S or N; most preferably N. X14, X23 and X50 may of course exhibit different amino acid substitutions, for example X14 may be R while X23 and X50 are S; or X14 and X23 are S, while X50 is N; X14 may be R while X23 is S and X50 is N etc. Any other combination conceivable is also contemplated by the present invention. Conservative amino acid substitutions are preferred. Particularly preferred is a substitute of a serine residue for the cysteine residue. Thus, in particularly preferred examples of the present invention X14 is S, X23 is S or X50 is S. Of course, it is also possible that X14 and X23 are S, or that X14 and X50 are S, or that X23 and X50 are S. X14, X23 and X50 may also all three be S. Absence of one or more or even of all of these cysteine residue has the advantage that the risk of aggregation of the polypeptide according to the present invention, e.g. by undesired disulfide bridge formation, is reduced, and is thus an preferred embodiment of the present invention.

Aside of the dispensability of the above referenced cysteine residues, the authors of WO 2015/071436 have also elucidated that various other residues in the sequence of SEQ ID NO:8 are also not essential and, moreover, may be replaced by other residues, thereby increasing for instance the temperature stability of the inventive polypeptide. Examples for such substitutions are X82I, X122M, X149P; X154T, X160T, X167L, X179F, X180E, X186Y, X206V, X206N, X212N, X230Y and X232T. These substitutions may be present alone or in any combination. A typical combination is the combination of X122M and X160T. Other examples of combinations are, without being limited thereto, X82I, X206V plus X232T; X82I, X122M, X160T, X206V, plus X232T; X82I, X122M, X160T, X206N, plus X232T; X82I, X122M, X206V, plus X232T; X82I, X122M, X149P, X160T, X206V, plus X232T; X82I, X122M, X160T, X180E, X206V, plus X232T; X82I, X122M, X160T, X186Y, X206V, plus X232T; X82I, X122M, X160T, X206V, X230Y, plus X232T; X82I, X122M, X149P, X206V, plus X232T; X82I, X122M, X149P, X160T, X206V, plus X232T; X82I, X122M, X149P, X206V, plus X232T; X82I, X122M, X149P, X167L, X206V, plus X232T; X82I, X122M, X149P, X179F, X206V, plus X232T; X82I, X122M, X149P, X206V, X212N plus X232T; X82I, X122M, X149P, X206V, X224Q plus X232T; X82I, X122M, X149P, X154T, X206V, plus X232T etc. Of course, this second type of amino acid modifications may be combined with the above mentioned cysteine replacements in any type of combination conceivable. Examples of such combinations are, without being limited thereto, X14S, X50S, X122M and X160T; X14S, X50S, X82I, X122M, X160T, X206V, and X232T; X14S, X50S, X82I, X122M, X160T, X206N, and X232T; X14S, X50S, X82I, X122M, X206V, and X232T; X14S, X50S, X82I, X122M, X149P, X160T, X206V, and X232T; X14S, X50S, X82I, X122M, X160T, X180E, X206V, and X232T; X14S, X50S, X82I, X122M, X160T, X186Y, X206V, and X232T; X14S, X50S, X82I, X122M, X160T, X206V, X230Y, and X232T; X14R, X50S, X82I, X122M, X160T, X206V, and X232T; X14S, X50N, X82I, X122M, X160T, X206V, and X232T; X14R, X50S, X82I, X122M, X149P, X206V, and X232T; X14R, X50S, X82I, X122M, X149P, X160T, X206V, and X232T; X14R, X50N, X82I, X122M, X149P, X206V, and X232T; X14R, X50N, X82I, X122M, X149P, X167L, X206V, and X232T; X14R, X50N, X82I, X122M, X149P, X179F, X206V, and X232T; X14R, X50N, X82I, X122M, X149P, X206V, X212N, and X232T; X14R, X50N, X82I, X122M, X149P, X206V, X224Q and X232T; X14R, X50N, X82I, X122M, X149P, X154T, X206V, and X232T; etc.

Examples of particularly preferred derivatives of SEQ ID NO:2 are for instance SEQ ID NO:9; SEQ ID NO:10; SEQ ID NO:11; SEQ ID NO:12; SEQ ID NO:13; SEQ ID NO:14; SEQ ID NO:15; SEQ ID NO:16; SEQ ID NO:17; SEQ ID NO:18; SEQ ID NO:19; SEQ ID NO:20; SEQ ID NO:21; SEQ ID NO:22; SEQ ID NO:23; SEQ ID NO:24; SEQ ID NO:25; SEQ ID NO:26; SEQ ID NO:27; SEQ ID NO:28; SEQ ID NO:29; and SEQ ID NO:30 (and corresponding sequences with N-terminal methionine). A particularly preferred derivative of SEQ ID NO:2 is SEQ ID NO:13.

Suitable sequences for the first amino acid sequence of the polypeptide according to the present invention are for example SEQ ID NO:31, SEQ ID NO:32, and SEQ ID NO:33. Other examples for the first amino acid sequence of the polypeptide according to the present invention include SEQ ID NO:34 or SEQ ID NO:35. A further example is the amino acid sequence of SEQ ID NO:7. In certain embodiments of the invention, in particular where the polypeptide comprises an amino acid sequence according to SEQ ID NO:3, a derivative of SEQ ID NO:3 as defined herein, an amino acid sequence according to SEQ ID NO:4, and/or a derivative of SEQ ID NO:4 as defined herein, the amino acid sequence according to SEQ ID NO:5 is also a suitable sequence for the first amino acid sequence of the polypeptide according to the present invention.

In preferred embodiments of the invention the the second amino acid sequence is selected from the group consisting of:
i) an amino acid sequence according to SEQ ID NO:3;
ii) a derivative of SEQ ID NO:3 exhibiting at least 77% sequence identity with SEQ ID NO:3;
iii) an amino acid sequence according to SEQ ID NO:4; and
iv) a derivative of SEQ ID NO:4 exhibiting at least 80% sequence identity with SEQ ID NO:4.

As mentioned previously, a polypeptide of the present invention comprising the amino acid sequence according to SEQ ID NO:4 may not comprise in parallel the sequence of SEQ ID NO:6. Preferably, such polypeptide according to the present invention does not comprise the sequence of SEQ ID NO:2. In particular, a polypeptide according to the present invention does not comprise the sequence of SEQ ID NO:36. This proviso applies for the polypeptides of the invention per se, but not necessarily for other aspects of the present invention, e.g. methods and uses employing such polypeptide, in particular when dealing with Salmonella bacteria. However, said proviso may of course also apply to the other aspects, e.g. the methods and uses of such polypeptide.

Derivatives of SEQ ID NO:3 exhibit preferably at least 81%, at least 86%, at least 90%, or at least 95% sequence identity with SEQ ID NO:3. Derivatives of SEQ ID NO:3 are preferably fragments of SEQ ID NO:3 and/or exhibit conservative amino acid substitutions vis-à-vis SEQ ID NO:3. Particularly preferred derivatives of SEQ ID NO:3 are fragments of SEQ ID NO:3. For example, a 17-mer fragment (SEQ ID NO:37) of SEQ ID NO:3 has been described in the art (Monteiro et al, Mol Pharm., 2015; 12(8):2904-11) which exhibits improved selectivity.

Derivatives of SEQ ID NO:4 exhibit preferably at least 83%, at least 86%, at least 90%, at least 93%, or at least 96% sequence identity with SEQ ID NO:4. Derivatives of SEQ ID NO:4 are preferably fragments of SEQ ID NO:4 and/or exhibit conservative amino acid substitutions vis-à-vis SEQ ID NO:4.

Preferred combinations of first and second amino acid sequence in the polypeptide according to the present invention are polypeptides, wherein the first amino acid sequence is SEQ ID NO:1 or said derivative thereof, and wherein the second amino acid sequence is SEQ ID NO:3 or said derivative thereof. A preferred combination is also wherein the first amino acid sequence is SEQ ID NO:1 or said derivative thereof, and wherein the second amino acid sequence is SEQ ID NO:4 or said derivative thereof. A further preferred combination is where the first amino acid sequence is SEQ ID NO:2 or said derivative thereof, and wherein the second amino acid sequence is SEQ ID NO:3 or said derivative thereof. Preferred are also combinations, wherein the first amino acid sequence is SEQ ID NO:2 and wherein the second amino acid sequence is said derivative of SEQ ID NO:4, or wherein the first amino acid sequence is said derivative of SEQ ID NO:2, and the second amino acid sequence is SEQ ID NO:4 or said derivative thereof. Similarly preferred combinations are those, wherein the first amino acid sequence is SEQ ID NO:2 or said derivative thereof, and wherein the second amino acid sequence is SEQ ID NO:4 or said derivative thereof, in particular a derivative according to SEQ ID NO:13.

Particularly preferred combinations are wherein
i) the first amino acid sequence is SEQ ID NO: 1 and the second amino acid sequence is SEQ ID NO:3,
ii) the first amino acid sequence is SEQ ID NO: 1 and the second amino acid sequence is SEQ ID NO:4, and
iii) wherein the first amino acid sequence is SEQ ID NO: 13 and the second amino acid sequence is SEQ ID NO:4.

In other embodiments of the invention, where the second amino acid sequence of the inventive polypeptide is neither SEQ ID NO:3 nor SEQ ID NO:4, the second amino acid sequence is selected from the group consisting of an antimicrobial peptide, amphiphatic peptide, cationic peptide, polycationic peptide, hydrophobic peptide, sushi peptide or defensin.

Examples for cationic/polycationic amino acid sequences are listed in the following table.

TABLE 1

| | | SEQ ID |
|---|---|---|
| Amino acid sequence | Length | NO: |
| KRKKRK | 6 | 38 |
| KRXKR | 5 | 39 |
| KRSKR | 5 | 40 |
| KRGSG | 5 | 41 |
| KRKKRKKRK | 9 | 42 |
| RRRRRRRRR | 9 | 43 |
| KKKKKKKK | 8 | 44 |
| KRKKRKKRKK | 10 | 45 |
| KRKKRKKRKKRK | 12 | 46 |
| KRKKRKKRKKRKKR | 14 | 47 |
| KKKKKKKKKKKKKKKK | 16 | 48 |
| KRKKRKKRKKRKKRKKRK | 18 | 49 |
| KRKKRKKRKKRKKRKKRKK | 19 | 50 |
| RRRRRRRRRRRRRRRRRRR | 19 | 51 |
| KKKKKKKKKKKKKKKKKKK | 19 | 52 |
| KRKKRKKRKRSKRKKRKKRK | 20 | 53 |
| KRKKRKKRKRSKRKKRKKRKK | 21 | 54 |
| KRKKRKKRKKRKKRKKRKKRK | 21 | 55 |
| KRKKRKKRKRGSGKRKKRKKRK | 22 | 56 |
| KRKKRKKRKRGSGSGKRKKRKKRK | 24 | 57 |
| KRKKRKKRKKRKKRKKRKKRKKRKK | 25 | 58 |
| KRKKRKKRKRSKRKKRKKRKRSKRKKRKKRK | 31 | 59 |
| KRKKRKKRKRGSGSGKRKKRKKRKGSGSGKR | 38 | 60 |
| KKRKKRK | | |
| KRKKRKKRKKRKKRKKRKKRKKRKKRKKRKK | 39 | 61 |
| RKKRKKRK | | |
| KRKKRKKRKRSKRKKRKKRKRSKRKKRKKRK | 42 | 62 |
| RSKRKKRKKRK | | |

Examples for antimicrobial amino acid sequences which may be used in carrying out the present invention are listed in the following table.

TABLE 2

| | | SEQ |
|---|---|---|
| | | ID |
| Peptide | Sequence | NO |
| LL-37 | LLGDFFRKSKEKIGKEFKRIVQRIKDFLRNL | 63 |
| | VPRTES | |
| SMAP-29 | RGLRRLGRKIAHGVKKYGPTVLRIIRIAG | 64 |
| Indolicidin | ILPWKWPWWPWRR | 65 |
| Protegrin | RGGRLCYCRRRFCVCVGR | 66 |
| Cecropin P1 | SWLSKTAKKLENSAKKRISEGIAIAIQGGPR | 67 |
| Magainin | GIGKFLHSAKKFGKAFVGEIMNS | 68 |
| Pleurocidin | GWGSFFKKAAHVGKHVGKAALTHYL | 69 |
| Cecropin A | GGLKKLGKKLEGAGKRVFNAAEKALPVVAGA | 70 |
| (A. aegypti) | KALRK | |
| Cecropin A | GWLKKIGKKIERVGQHTRDATIQGLGIPQQA | 71 |
| (D. | ANVAATARG | |
| melanogaster) | | |
| Buforin II | TRSSRAGLQFPVGRVHRLLRK | 72 |
| Sarcotoxin IA | GWLKKIGKKIERVGQHTRDATIQGLGIAQQA | 73 |
| | ANVAATAR | |
| Apidaecin | ANRPVYIPPPRPPHPRL | 74 |
| Ascaphine 5 | GIKDWIKGAAKKLIKTVASHIANQ | 75 |
| Nigrocine 2 | GLLSKVLGVGKKVLCGVSGLVC | 76 |
| Pseudin 1 | GLNTLKKVFQGLHEAIKLINNHVQ | 77 |
| Ranalexin | FLGGLIVPAMICAVTKKC | 78 |
| Melittin | GIGAVLKVLTTGLPALISWIKRKRQQ | 79 |
| Lycotoxin 1 | IWLTALKFLGKHAAKKLAKQQLSKL | 80 |
| Parasin 1 | KGRGKQGGKVRAKAKTRSS | 81 |
| Buforin I | AGRGKQGGKVRAKAKTRSSRAGLQFPVGRVH | 82 |
| | RLLRKGNY | |
| Dermaseptin 1 | ALWKTMLKKLGTMALHAGKAALGAAADTISQ | 83 |
| | GTQ | |
| Bactenecin 1 | RLCRIVVIRVCR | 84 |
| Thanatin | GSKKPVPIIYCNRRTGKCQRM | 85 |
| Brevinin 1T | VNPIILGVLPKVCLITKKC | 86 |
| Ranateurin 1 | SMLSVLKNLGKVGLGFVACKINIKQC | 87 |
| Esculentin 1 | GIFSKLGRKKIKNLLISGLKNVGKEVGMDVV | 88 |
| | RTGIKIAGCKIKGEC | |
| Tachyplesin | RWCFRVCYRGICYRKCR | 89 |
| Androctonin | RSVCRQIKICRRRGGCYYKCTNRPY | 90 |
| alpha- | DCYCRIPACIAGERRYGTCIYQGRLWAFCC | 91 |
| defensin | | |
| beta- | NPVSCVRNKGICVPIRCPGSMKQIGTCVGRA | 92 |
| defensin | VKCCRKK | |
| theta- | GFCRCLCRRGVCRCICTR | 93 |
| defensin | | |
| defensin | ATCDLLSGTGINHSACAAHCLLRGNRGGYCN | 94 |
| (sapecin A) | GKAVCVCRN | |
| Thionin | TTCCPSIVARSNFNVCRIPGTPEAICATYTG | 95 |
| (crambin) | CIIIPGATCPGDYAN | |
| defensin | QKLCQRPSGTWSGVCGNNNACKNQCIRLEKA | 96 |
| from | RHGSCNYVFPAHCICYFPC | |
| radish | | |
| Drosomycin | DCLSGRYKGPCAVWDNETCRRVCKEEGRSSG | 97 |
| | HCSPSLKCWCEGC | |
| Hepcidin | DTHFPICIFCCGCCHRSKCGMCCKT | 98 |
| Bac 5 | RFRPPIRRPPIRPPFYPPFRPPIRPPIFPPI | 99 |
| | RPPFRPPLGRPFP | |
| PR-39 | RRRPRPPYLPRPRPPPFFPPRLPPRIPPGFP | 100 |
| | PRFPPRFP | |
| Pyrrhocoricin | VDKGSYLPRPTPPRPIYNRN | 101 |
| Histatin 5 | DSHAKRHHGYKRKFHEKHHSHRGY | 102 |
| ECP19 | RPPQFTRAQWFAIQHISLN | 103 |
| MSI-594 | GIGKFLKKAKKGIGAVLKVLTTG | 104 |
| TL-ColM | METLTVHAPSPSTNLPSYGNGAFSLSAPHVP | 105 |
| | GAGP | |
| SBO | KLKKIAQKIKNFFAKLVA | 106 |
| Macedocin | GKNGVFKTISHECHLNTWAFLATCCS | 107 |
| Macedocin | GKNGVFKTISHECHLNTWAFLA | 108 |
| (Trunc) | | |
| D16 | ACKLKSLLKTLSKAKKKKLKTLLKALSK | 109 |
| CPF-C1 | GFGSLLGKALRLGANVL | 110 |
| TL-ColM(-Met) | ETLTVHAPSPSTNLPSYGNGAFSLSAPHVPG | 111 |
| | AGP | |
| TM-174E | LISKGWPYLLVVVLGATIYFWGNSNG | 112 |
| ECP45 | RPPQFTRAQWFAIQHISLNPPRCTIAMRAIN | 113 |
| | NYRWRCKNQNTFLR | |
| ColicinE3_1- | SGGDGRGHNTGAHSTSGNINGGPTGLGVGGG | 114 |
| 51 (S37F) | ASDGFGWSSENNPWGGGSG | |
| ColicinE3_1- | SGGDGRGHNTGAHSTSGNINGGPTGLGVGGG | 115 |
| 69(S37F) | ASDGFGWSSENNPWGGGSGSGIHWGGGSGHG | |
| | NGGGNG | |
| ColicinD_1-53 | SDYEGSGPTEGIDYGHSMVVWPSTGLISGGD | 116 |
| | VKPGGSSGIAPSMPPGWGDYS | |
| MSI-78 | GIGKFLKKAKKFGKAFVKILKK | 3 |
| Cathelicidin- | KFFRKLKKSVKKRAKEFFKKPRVIGVSIPF | 4 |
| BF | | |

The second amino acid sequence stretch may be a sushi peptide which is described by Ding J L, Li P, Ho B Cell Mol Life Sci. 2008 April; 65(7-8):1202-19. The Sushi peptides: structural characterization and mode of action against Gram-negative bacteria. Especially preferred is the sushi 1 peptide according to SEQ ID NO: 117. Other preferred sushi peptides are sushi peptides S1 and S3 and multiples thereof (Tan et al, FASEB J. 2000 September; 14(12):1801-13).

Preferred hydrophobic peptides are Walmagh1 having the amino acid sequence according to SEQ ID NO: 118 and the hydrophobic peptide having the amino acid sequence Phe-Phe-Val-Ala-Pro (SEQ ID NO: 119).

Preferred amphiphatic peptides are α4-helix of T4 lysozyme according to SEQ ID NO: 120 and WLBU2-Variant having the amino acid sequence according to SEQ ID NO: 121 and Walmagh 2 according to SEQ ID NO: 122.

With respect to the arrangement of first and second amino acid sequence within the inventive polypeptide it is preferred if the second amino acid sequence is situated N-terminal of the first amino acid sequence. Preferably, the first and second amino acid sequence are linked to each other directly or via a short linker of 1 to 10 amino acid residues, preferably 1 to 5 amino acid residues, even more preferably 1 to 2 amino acids. Linker sequences are preferably flexible sequences, comprising one or more glycine residues. An example for such linker is the sequence GGGGS (SEQ ID NO: 123).

In particular in cases where the inventive polypeptide is to be recombinantly expressed by a host cell, it is preferred if the inventive polypeptide comprises a methionine residue at the N-terminus.

The inventive polypeptide may comprise additionally one or more tag sequences. Such tag sequence may for example be located at the N- or C-terminus of the inventive polypeptide. In a preferred embodiment, the one or more tag sequence is located on the C-terminal side of the polypeptide, e.g. directly at the C-terminus. The one or more tag sequences may be linked for example directly or via a short linker to the rest of the inventive polypeptide (see above). Numerous examples for tags are known in the art, some of which have already been mentioned above. In the context of the present invention a particularly preferred tag sequence is a His-tag, preferably a His tag according to SEQ ID NO: 124.

The length of the polypeptide according to present invention is in principle not limited, but preferably the length will not be excessively large. Preferably, a polypeptide according to the present invention has an overall length not exceeding about 320 amino acids, preferably not exceeding about 310 amino acids.

A preferred polypeptide according to the invention comprises SEQ ID NO: 125, such as a polypeptide comprising SEQ ID NO: 126 or comprising SEQ ID NO: 127. Also contemplated are derivatives of SEQ ID NO: 125, SEQ ID NO: 126 and SEQ ID NO: 127, e.g. each exhibiting at least 80% sequence identity to the respective reference sequence.

A further preferred polypeptide according to the invention comprises SEQ ID NO: 128, such as a polypeptide comprising SEQ ID NO: 129 or comprising SEQ ID NO: 130. Also contemplated are derivatives of SEQ ID NO: 128, SEQ ID NO: 129 and SEQ ID NO: 130, e.g. each exhibiting at least 80% sequence identity to the respective reference sequence.

A further preferred polypeptide according to the invention comprises SEQ ID NO: 131, such as a polypeptide comprising SEQ ID NO: 132 or comprising SEQ ID NO: 133. Also contemplated are derivatives of SEQ ID NO: 131, SEQ ID NO: 132 and SEQ ID NO: 133, e.g. each exhibiting at least 80% sequence identity to the respective reference sequence.

A further polypeptide according to the invention comprises SEQ ID NO: 134, such as a polypeptide comprising SEQ ID NO: 135 or comprising SEQ ID NO: 136. Also contemplated are derivatives of SEQ ID NO: 134, SEQ ID NO: 135 and SEQ ID NO: 136, e.g. each exhibiting at least 80% sequence identity to the respective reference sequence.

Further examples of inventive polypeptides are polypeptides comprising any of the sequences selected from the group consisting of SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148 and derivatives of these sequences, e.g. each exhibiting at least 80% sequence identity to the respective reference sequence

A polypeptide according to the present invention is preferably characterized by the ability to degrade the peptidoglycan of Salmonella bacteria. The peptidoglycan degrading activity can be measured by assays well known in the art, e.g. by muralytic assays in which the outer membrane of gram negative bacteria such as Salmonella bacteria is permeabilized or removed (e.g. with chloroform) to allow the putative enzyme access to the peptidoglycan layer. If the enzyme is active, degradation of the peptidoglycan layer will lead to a drop of turbidity, which can be measured photometrically (see for example Briers et al., J. Biochem. Biophys Methods 70: 531-533, (2007).

The present invention does also relate to nucleic acids encoding one or more inventive polypeptides of the present invention. The inventive nucleic acid may take all forms conceivable for a nucleic acid. In particular the nucleic acids according to the present invention may be RNA, DNA or hybrids thereof. They may be single-stranded or double-stranded. The may have the size of small transcripts or of entire genomes, such as a bacteriophage genome. As used herein, a nucleic acid encoding one or more inventive polypeptides of the present invention may be a nucleic acid reflecting the sense strand. Likewise, the antisense strand is also encompassed. The nucleic acid may encompass a heterologous promoter for expression of the inventive polypeptide.

A nucleic acid according to the present invention comprises a first nucleic acid sequence encoding an endolysin, and a second nucleic acid sequence, wherein the second nucleic acid sequence encodes an antimicrobial peptide, cationic peptide, hydrophobic peptide, amphiphatic peptide or sushi peptide, and wherein the nucleic acid according to the present invention comprises at least one sequence selected from the following group of sequences:
i) a nucleic acid sequence according to SEQ ID NO: 149;
ii) a derivative of SEQ ID NO:149 encoding the same polypeptide as SEQ ID NO: 149,
iii) a nucleic acid sequence according to SEQ ID NO: 150;
iv) a derivative of SEQ ID NO:150 encoding the same polypeptide as SEQ ID NO: 150;
v) a nucleic acid sequence according to SEQ ID NO: 151; and
vi) a derivative of SEQ ID NO:151 encoding the same polypeptide as SEQ ID NO:151.

Preferred nucleic acids according to the present invention comprise the nucleic acid sequence according to SEQ ID NO:149 as first nucleic acid sequence and SEQ ID NO:150 as second nucleic acid sequence, or comprise SEQ ID NO: 152 as first nucleic acid sequence and SEQ ID NO:151 as second nucleic acid sequence. Particularly preferred nucleic acid sequences comprise SEQ ID NO:153 or SEQ ID NO:154.

In a further aspect, the present invention relates to a vector, which comprises a nucleic acid according to the present invention. Such vector may for example be an expression vector allowing for expression of an inventive polypeptide. Said expression may be constitutive or inducible. The vector may also be a cloning vector comprising the nucleic acid sequence of an inventive polypeptide for cloning purposes.

In a further aspect, the present invention relates to a host cell comprising a polypeptide according to the present invention, a nucleic acid according to the present invention, and/or a vector according to the present invention. The host cells may be selected in particular from the group consisting of bacterial cells and yeast cells. Particularly preferred host cells are E. coli cells.

In a further aspect, the present invention relates to composition comprising a polypeptide according to the present invention, a nucleic acid according to the present invention, a vector according to the present invention, and/or a host cell according to the present invention. Preferred compositions comprise the polypeptide according to the present invention. Preferably, a composition according to the present invention comprises a pharmaceutical acceptable diluent, excipient or carrier. Such composition may be a pharmaceutical composition. A composition according to the present invention may also be a feed additive.

In a further aspect the present invention relates to a polypeptide according to the present invention, a nucleic acid according to the present invention, a vector according to the present invention, a host cell according to the present invention, and/or a composition according the present invention for use in a method for treatment of the human or animal body by surgery or therapy or in diagnostic methods practiced on the human or animal body.

The present invention also relates to a polypeptide (and likewise a nucleic acid encoding such polypeptide, a vector comprising such nucleic acid, a host cell comprising such nucleic acid or vector, and/or a composition comprising such polypeptide, nucleic acid, vector, and/or host cell) for use in a method of treatment or prevention of infections caused by bacteria of the genus Salmonella, wherein the polypeptide comprises a first and a second amino acid sequence, wherein the first amino acid sequence is an endolysin, and wherein the second amino acid sequence is an antimicrobial peptide, amphiphatic peptide, cationic peptide, hydrophobic peptide, sushi peptide or defensin, and wherein the polypeptide comprises at least one sequence selected from the following group of sequences:
i) an amino acid sequence according to SEQ ID NO: 1;
ii) a derivative of SEQ ID NO: 1 exhibiting at least 80% sequence identity with SEQ ID NO:1,
iii) an amino acid sequence according to SEQ ID NO:2;
iv) a derivative of SEQ ID NO:2 exhibiting at least 80% sequence identity with SEQ ID NO:2,
v) an amino acid sequence according to SEQ ID NO:3;
vi) a derivative of SEQ ID NO:3 exhibiting at least 77% sequence identity with SEQ ID NO:3;
vii) an amino acid sequence according to SEQ ID NO:4; and
viii) a derivative of SEQ ID NO:4 exhibiting at least 80% sequence identity with SEQ ID NO:4.

Disclosure set out above for the inventive polypeptide is contemplated for the polypeptide (and the nucleic acid encoding such polypeptide, the vector comprising such nucleic acid, the host cell comprising such nucleic acid or vector, and/or the composition comprising such polypeptide, nucleic acid, vector, and/or host cell) for use in a method of treatment or prevention of infections caused by bacteria of the genus Salmonella as well. In particular, disclosure detailing SEQ ID NO:2 and its derivatives is contemplated as embodiments for the polypeptide for use in a method of treatment or prevention of infections caused by bacteria of the genus Salmonella (see for instance examples of particularly preferred derivatives of SEQ ID NO:2 above, such as SEQ ID NO: 13). As mentioned previously, the provisos regarding the polypeptide of the invention (polypeptide may not comprise the sequence of SEQ ID NO:5, if the polypeptide of the invention does not comprise in parallel an amino acid sequence according to SEQ ID NO:3, a derivative of SEQ ID NO:3 as defined herein, an amino acid sequence according to SEQ ID NO:4, and/or a derivative of SEQ ID NO:4 as defined herein; polypeptide may not comprise in parallel the sequence of SEQ ID NO:6, if the polypeptide comprises SEQ ID NO:4) do not necessarily but preferably apply for this aspect of the present invention. Thus, in some embodiments, the polypeptide for use is an inventive polypeptide as set forth above, i.e. the present invention relates to a polypeptide according to the present invention, a nucleic acid according to the present invention, a vector according to the present invention, a host cell according to the present invention, and/or a composition according the present invention for use in a method of treatment or prevention of infections caused by bacteria of the genus Salmonella.

The present invention also relates to a method of treatment or prevention of infections caused bacteria of the genus Salmonella in a subject, the method comprising contacting said subject with a polypeptide (or likewise a nucleic acid encoding such polypeptide, a vector comprising such nucleic acid, a host cell comprising such nucleic acid or vector, and/or a composition comprising such polypeptide, nucleic acid, vector, and/or host cell), wherein the polypeptide comprises a first and a second amino acid sequence, wherein the first amino acid sequence is an endolysin, and wherein the second amino acid sequence is an antimicrobial peptide, amphiphatic peptide, cationic peptide, hydrophobic peptide, sushi peptide or defensin, and wherein the polypeptide comprises at least one sequence selected from the following group of sequences:
i) an amino acid sequence according to SEQ ID NO: 1;
ii) a derivative of SEQ ID NO: 1 exhibiting at least 80% sequence identity with SEQ ID NO:1,
iii) an amino acid sequence according to SEQ ID NO:2;
iv) a derivative of SEQ ID NO:2 exhibiting at least 80% sequence identity with SEQ ID NO:2,
v) an amino acid sequence according to SEQ ID NO:3;
vi) a derivative of SEQ ID NO:3 exhibiting at least 77% sequence identity with SEQ ID NO:3;
vii) an amino acid sequence according to SEQ ID NO:4; and
viii) a derivative of SEQ ID NO:4 exhibiting at least 80% sequence identity with SEQ ID NO:4.

Again, disclosure set out above for the inventive polypeptide is contemplated for the polypeptide (and the nucleic acid encoding such polypeptide, the vector comprising such nucleic acid, the host cell comprising such nucleic acid or vector, and/or the composition comprising such polypeptide, nucleic acid, vector, and/or host cell) to be used in said method of treatment as well. In particular, disclosure detailing SEQ ID NO:2 and its derivatives is contemplated as embodiments for the polypeptide to be used in the method of treatment or prevention of infections caused by bacteria of the genus Salmonella (see for instance examples of particularly preferred derivatives of SEQ ID NO:2 above, such as SEQ ID NO:13). As mentioned previously, the provisos regarding the polypeptide of the invention (polypeptide may not comprise the sequence of SEQ ID NO:5, if the polypeptide of the invention does not comprise in parallel an amino acid sequence according to SEQ ID NO:3, a derivative of SEQ ID NO:3 as defined herein, an amino acid sequence according to SEQ ID NO:4, and/or a derivative of SEQ ID NO:4 as defined herein; polypeptide may not comprise in parallel the sequence of SEQ ID NO:6, if the polypeptide comprises SEQ ID NO:4) do not necessarily but preferably apply for this aspect of the present invention. Thus, in some embodiments, the polypeptide to be used in the inventive method of treatment is an inventive polypeptide as set forth above, i.e. the present invention relates to a method of treatment or prevention of infections caused bacteria of the genus Salmonella in a subject, the method comprising contacting said subject with a polypeptide according to the present invention, a nucleic acid according to the present invention, a vector according to the present invention, a host cell according to the present invention, and/or a composition according the present invention.

In a further aspect the present invention relates to the use of a polypeptide (or likewise a nucleic acid encoding such polypeptide, a vector comprising such nucleic acid, a host cell comprising such nucleic acid or vector, and/or a composition comprising such polypeptide, nucleic acid, vector, and/or host cell) as an antimicrobial in food, as an antimicrobial in feed, as an antimicrobial in cosmetics, or as disinfecting agent, wherein the polypeptide comprises a first and a second amino acid sequence, wherein the first amino acid sequence is an endolysin, and wherein the second amino acid sequence is an antimicrobial peptide, amphiphatic peptide, cationic peptide, hydrophobic peptide, sushi peptide or defensin, and wherein the polypeptide comprises at least one sequence selected from the following group of sequences:
i) an amino acid sequence according to SEQ ID NO: 1;
ii) a derivative of SEQ ID NO: 1 exhibiting at least 80% sequence identity with SEQ ID NO:1,
iii) an amino acid sequence according to SEQ ID NO:2;
iv) a derivative of SEQ ID NO:2 exhibiting at least 80% sequence identity with SEQ ID NO:2,
v) an amino acid sequence according to SEQ ID NO:3;
vi) a derivative of SEQ ID NO:3 exhibiting at least 77% sequence identity with SEQ ID NO:3;
vii) an amino acid sequence according to SEQ ID NO:4; and
viii) a derivative of SEQ ID NO:4 exhibiting at least 80% sequence identity with SEQ ID NO:4.

Again, the provisos regarding the polypeptide of the invention (see above) do not necessarily but preferably apply for this aspect of the present invention. Thus, the present invention does also relate to a polypeptide according to the present invention, a nucleic acid according the present invention, a vector according to the present invention, a host cell according to the present invention, and/or a composition according to the present invention as an antimicrobial in food, as an antimicrobial in feed, as an antimicrobial in cosmetics, or as disinfecting agent.

In a further aspect the present invention relates to the use of a polypeptide (and likewise a nucleic acid encoding such polypeptide, a vector comprising such nucleic acid, a host cell comprising such nucleic acid or vector, and/or a composition comprising such polypeptide, nucleic acid, vector, and/or host cell) for controlling (e.g. non-therapeutically) the growth of bacteria of the genus Salmonella in animals, in particular in livestock, companion animal and/or aquaculture, wherein the polypeptide comprises a first and a second amino acid sequence, wherein the first amino acid sequence is an endolysin, and wherein the second amino acid sequence is an antimicrobial peptide, amphiphatic peptide, cationic peptide, hydrophobic peptide, sushi peptide or defensin, and wherein the polypeptide comprises at least one sequence selected from the following group of sequences:
i) an amino acid sequence according to SEQ ID NO: 1;
ii) a derivative of SEQ ID NO: 1 exhibiting at least 80% sequence identity with SEQ ID NO:1,
iii) an amino acid sequence according to SEQ ID NO:2;
iv) a derivative of SEQ ID NO:2 exhibiting at least 80% sequence identity with SEQ ID NO:2,
v) an amino acid sequence according to SEQ ID NO:3;
vi) a derivative of SEQ ID NO:3 exhibiting at least 77% sequence identity with SEQ ID NO:3;
vii) an amino acid sequence according to SEQ ID NO:4; and
viii) a derivative of SEQ ID NO:4 exhibiting at least 80% sequence identity with SEQ ID NO:4.

The provisos regarding the polypeptide of the invention (see above) do not necessarily but preferably apply for this aspect of the present invention as well. Disclosure set out above for the inventive polypeptide is contemplated for the polypeptide (and the nucleic acid encoding such polypeptide, the vector comprising such nucleic acid, the host cell comprising such nucleic acid or vector, and/or the composition comprising such polypeptide, nucleic acid, vector, and/or host cell) to be used for controlling (e.g. non-therapeutically) the growth of bacteria of the genus Salmonella in animals (in particular in livestock, companion animal and/or aquaculture) as well. In particular, disclosure detailing SEQ ID NO:2 and its derivatives is contemplated as embodiments for the polypeptide to be used for controlling (non-therapeutically) the growth of bacteria of the genus Salmonella in animals such as livestock, companion animal and/or aquaculture (see for instance examples of particularly preferred derivatives of SEQ ID NO:2 above, such as SEQ ID NO: 13). In some embodiments, the polypeptide to be used for controlling (e.g. non-therapeutically) the growth of bacteria of the genus Salmonella in animals is an inventive polypeptide as set forth above, i.e. the present invention relates to the use of a polypeptide according to the present invention, a nucleic acid according to the present invention, a vector according to the present invention, a host cell according to the present invention, and/or a composition according the present invention for controlling (e.g. non-therapeutically) the growth of bacteria of the genus Salmonella in animals, in particular in livestock, companion animal and/or aquaculture.

In a further aspect the present invention relates to a method of controlling the growth of bacteria of the genus Salmonella in animals, in particular in livestock, companion animal and/or aquaculture, the method comprising contacting livestock, companion animal and/or aquaculture with a polypeptide (or a nucleic acid encoding such polypeptide, a vector comprising such nucleic acid, a host cell comprising such nucleic acid or vector, and/or a composition comprising such polypeptide, nucleic acid, vector, and/or host cell), wherein the polypeptide comprises a first and a second amino acid sequence, wherein the first amino acid sequence is an endolysin, and wherein the second amino acid sequence is an antimicrobial peptide, amphiphatic peptide, cationic peptide, hydrophobic peptide, sushi peptide or defensin, and wherein the polypeptide comprises at least one sequence selected from the following group of sequences:
i) an amino acid sequence according to SEQ ID NO: 1;
ii) a derivative of SEQ ID NO: 1 exhibiting at least 80% sequence identity with SEQ ID NO:1,
iii) an amino acid sequence according to SEQ ID NO:2;
iv) a derivative of SEQ ID NO:2 exhibiting at least 80% sequence identity with SEQ ID NO:2,
v) an amino acid sequence according to SEQ ID NO:3;
vi) a derivative of SEQ ID NO:3 exhibiting at least 77% sequence identity with SEQ ID NO:3;
vii) an amino acid sequence according to SEQ ID NO:4; and
viii) a derivative of SEQ ID NO:4 exhibiting at least 80% sequence identity with SEQ ID NO:4.

The provisos regarding the polypeptide of the invention (see above) do not necessarily but preferably apply for this aspect of the present invention as well. Again, disclosure set out above for the inventive polypeptide is contemplated for the polypeptide (and the nucleic acid encoding such polypeptide, the vector comprising such nucleic acid, the host cell comprising such nucleic acid or vector, and/or the composition comprising such polypeptide, nucleic acid, vector, and/or host cell) to be used in said method of controlling the growth of bacteria of the genus Salmonella in animals, in particular in livestock, companion animal and/or aquaculture, as well. In particular, disclosure detailing SEQ ID NO:2 and its derivatives is contemplated as embodiments for the polypeptide to be used in the method of controlling the growth of bacteria of the genus Salmonella in animals (see for instance examples of particularly preferred derivatives of SEQ ID NO:2 above, such as SEQ ID NO: 13). In some embodiments, the polypeptide to be used in the inventive method of treatment is an inventive polypeptide as set forth above, i.e. the present invention relates to a method of controlling the growth of bacteria of the genus Salmonella in animals, e.g. in livestock, companion animal and/or aquaculture, the method comprising contacting the animal (e.g. livestock, companion animal and/or aquaculture) with a polypeptide according to the present invention, a nucleic acid according to the present invention, a vector according to the present invention, a host cell according to the present invention, and/or the composition according to the present invention. Said inventive method of controlling the growth of bacteria of the genus Salmonella is preferably a non-therapeutic method, in particular where bacteria of the genus Salmonella are non-pathogenic for the respective animal (e.g. livestock, companion animal and/or aquaculture) or are only present in (for the animal) sub-pathogenic concentration, i.e. would not require medical treatment of said animal (i.e. livestock, companion animal and/or aquaculture).

### EXAMPLES

In the following, specific examples illustrating various embodiments and aspects of the invention are presented. However, the present invention shall not to be limited in scope by the specific embodiments described herein. Indeed, various modifications of the invention in addition to those described herein will become readily apparent to those skilled in the art from the foregoing description, accompanying figures and the examples below. All such modifications fall within the scope of the appended claims.

### Example 1: Data on Antibacterial Activity on S. Typhimurium LT2 from Prior Art Polypeptides Published in Briers et al, MBio. 2014 Jul. 1:5(4):e01379-14

Briers et al. published antibacterial activity of various fusion proteins for S. Typhimurium LT2 (see supplemental table 5). The data are summarized in table 3 (fusion variants of endolysin OBPgp279) and in table 4 (fusion variants of endolysin PVP-SE1gp146).

TABLE 3
Fusion variants of endolysin OBPgp279

| Fusion | Polypeptide | w/o EDTA | 0.5 mM EDTA |
|---|---|---|---|
| | | | 0.05 ± 0.05 |
| PCNP | LoGT-001 | 0.23 ± 0.03 | 0.91 ± 0.04 |
| MW1 | LoGT-002 | 0.15 ± 0.07 | 0.41 ± 0.08 |
| HPP | LoGT-003 | 0.09 ± 0.01 | 0.52 ± 0.04 |
| MW2 | LoGT-004 | 0.12 ± 0.06 | 0.36 ± 0.07 |
| α₄ | LoGT-005 | 0.17 ± 0.08 | 0.43 ± 0.09 |
| Parasin1 | LoGT-006 | 0.20 ± 0.14 | 0.60 ± 0.05 |
| Lycotoxin1 | LoGT-007 | 0.01 ± 0.11 | 0.51 ± 0.04 |

TABLE 4
Fusion variants of endolysin PVP-SE1gp146

| | | S. Typhimurium | |
|---|---|---|---|
| Fusion | Polypeptide | LT2 | 0.5 mM EDTA |
| | | | 0.05 ± 0.05 |
| PCNP | LoGT-008 | 0.30 ± 0.10 | 0.73 ± 0.30 |
| MW1 | LoGT-009 | 0.10 ± 0.07 | 0.27 ± 0.15 |
| HPP | LoGT-010 | 0.24 ± 0.16 | 0.47 ± 0.07 |
| MW2 | LoGT-011 | 0.12 ± 0.12 | 0.41 ± 0.25 |
| α₄ | LoGT-012 | 0.14 ± 0.12 | 0.47 ± 0.24 |
| Parasin1 | LoGT-013 | 0.04 ± 0.03 | 0.59 ± 0.38 |
| Lycotoxin1 | LoGT-014 | 0.20 ± 0.22 | 0.87 ± 0.28 |

### Example 2: Data on Antibacterial Activity of Selected Inventive Polypeptides on S. Typhimurium LT2

The inventors of the present invention have tested antibacterial activity of polypeptides comprising SEQ ID NO: 126 or SEQ ID NO: 129 (and further comprising a His-tag for purification reasons) on S. Typhimurium LT2. A first set of experiments was conducted in PBS buffer.

Briefly, exponentially growing cells of Salmonella Typhimurium LT2 were diluted 1:100 in 5 mM HEPES pH 7.4. Subsequently, 100 μl of cells were incubated for 30 min at room temperature with 50 μl protein solution dissolved in 1×PBS (170 mM NaCl, 3 mM KCl, 12.7 mM Na₂HPO₄, 2.2 mM KH₂PO₄, pH 7.4) and 50 μl of 5 mM HEPES pH 7.4 or 50 μL of EDTA (final concentration 0.5 mM) yielding a final protein concentration of 1.313 μM. Cells were serially diluted in 1×PBS after incubation and plated on LB agar. As a negative control, cells were incubated in the same buffer without any protein and additionally plated. Cell colonies were counted after incubation over night at 37° C. and the antibacterial activity was calculated in logarithmic units (=log 10No/Ni with N0=number of untreated colonies and Ni=number of treated colonies).

The results are shown in table 5 below.

TABLE 5
Antibacterial activity of inventive polypeptides in PBS buffer

| SEQ ID NO | w/o EDTA | 0.5 mM EDTA |
|---|---|---|
| SEQ ID NO: 126 | 2.75 | ≥4.00 |
| SEQ ID NO: 129 | 1.61 | 2.29 |

In a further set of experiments, antibacterial activity in HEPES-buffer on S. Typhimurium LT2 was determined.

Briefly, exponentially growing cells of Salmonella Typhimurium LT2 were diluted 1:100 in 5 mM HEPES pH 7.4. Subsequently, 100 μl of cells were incubated for 30 min at room temperature with 50 μl protein solution dissolved in 20 mM HEPES, 500 mM NaCl pH 7.4 and 50 μl of 5 mM HEPES pH 7.4 or 50 μL of EDTA (final concentration 0.5 mM) yielding a final protein concentration of 1.313 μM. Cells were serially diluted in 1×PBS after incubation and plated on LB agar. As a negative control, cells were incubated in the same buffer without any protein and additionally plated. Cell colonies were counted after incubation over night at 37° C. and the antibacterial activity was calculated in logarithmic units (=log 10No/Ni with N0=number of untreated colonies and Ni=number of treated colonies).

The results are shown in table 6 below.

TABLE 6
Antibacterial activity of inventive polypeptides in HEPES buffer

| SEQ ID NO | w/o EDTA | 0.5 mM EDTA |
|---|---|---|
| SEQ ID NO: 126 | 0.95 | 3.62 |
| SEQ ID NO: 129 | 1.24 | 1.61 |

In summary, antibacterial activity of the inventive polypeptides comprising the amino acids sequence of SEQ ID NO: 126 or SEQ ID NO: 129 on S. Typhimurium LT2 surprisingly exceeded the antibacterial activity achieved with similar compounds in the art significantly.

### Example 3: Data on Antibacterial Activity of Further Inventive Polypeptides on S. Typhimurium LT2

In another set of experiments, the inventors of the present invention have also tested antibacterial activity of further fusion proteins comprising the components of the polypeptides of the present invention. The polypeptides tested did comprise the sequences of SEQ ID NO: 138, SEQ ID NO: 141, SEQ ID NO: 132, SEQ ID NO: 144, and SEQ ID NO: 147 (and further comprising a His-tag for purification reasons) and were tested on S. Typhimurium LT2. The tested compounds all comprise at least one component which can be advantageously used according to the present invention (SEQ ID NO:1, SEQ ID NO:3 and (SEQ ID NO:4) for treating in particular Salmonella bacteria.

The experiment was done as set out in Example 2.

The results are shown in table 7 below.

TABLE 7
Antibacterial activity of inventive polypeptides in HEPES buffer

| SEQ ID NO | w/o EDTA | 0.5 mM EDTA |
|---|---|---|
| SEQ ID NO: 138 | 0.76 | 0.88 |
| SEQ ID NO: 141 | 1.71 | 1.47 |
| SEQ ID NO: 132 | 2.74 | >5 |
| SEQ ID NO: 144 | 0.15 | 3.24 |
| SEQ ID NO: 147 | 0.33 | 0.98 |

In summary, antibacterial activity of the inventive polypeptides comprising the amino acids sequence of SEQ ID NO: 138, SEQ ID NO: 141, SEQ ID NO: 132, SEQ ID NO: 144, and SEQ ID NO: 147 on S. Typhimurium LT2 reliably exceeded the antibacterial activity achieved with similar compounds in the art, indicating that the components used (SEQ ID NO:1, SEQ ID NO:3 and SEQ ID NO:4) all can provide for superior activity against Salmonella bacteria in comparison to the compounds of the prior art. Noteworthy, the polypeptide comprising two components of the invention (SEQ ID NO: 132) yielded again by far the best results.

### Example 4: Data on Antibacterial Activity of Selected Inventive Polypeptide on S. Typhimurium (DSM 17058)

The inventors of the present invention have tested antibacterial activity of an inventive polypeptide comprising SEQ ID NO: 135 (and further comprising a His-tag for purification reasons) on S. Typhimurium (DSM 17058).

The experiment was done as set out in Example 2.

The results are shown in table 8 below.

TABLE 8
Antibacterial activity of inventive polypeptides in HEPES buffer

| SEQ ID NO | w/o EDTA | 0.5 mM EDTA |
|---|---|---|
| SEQ ID NO: 135 | 3.91 | 3.88 |

Hence, antibacterial activity of the inventive polypeptide comprising the amino acid sequence of SEQ ID NO: 135 on S. Typhimurium (DSM 17058) exceeded the antibacterial activity achieved with similar compounds in the art, indicating that the endolysin component used (SEQ ID NO: 13) provides for superior activity against Salmonella bacteria in comparison to the respective components of the prior art polypeptides (i.e. endolysin components OBPgp279 and PVP-SE1gp146).

### Example 5: Data on Minimal Inhibitory Concentration of Selected Inventive Polypeptide on S. Typhimurium (DSM 17058)

The inventors of the present invention have tested antibacterial activity in terms of minimal inhibitory concentration (MIC) of an inventive polypeptide comprising SEQ ID NO: 132 (and further comprising a His-tag for purification reasons) on S. Typhimurium (DSM 17058).

Briefly, Bacteria were grown in (Luria-Bertani) medium and diluted 1:10 in Mueller-Hinton medium. At optical density OD₆₀₀ of about 0.6 bacteria were diluted in the same medium 1:10 followed by a 1:500 dilution in the same medium. Protein solutions were pipetted into a 96 well plate, using different concentrations of proteins and an end volume of 20 μl including 500 μM EDTA final concentration. 180 μl of bacterial cells or a medium (Mueller-Hinton) control were given to the 96 well plate and mixed. The plate was incubated for 18-22 hours at 37° C. and the bacterial growth was determined measuring the OD₆₀₀ values of the wells. The MIC which is the protein concentration of the well which showed the same OD₆₀₀ value as determined for the no-bacteria control.

The results in form of minimal inhibitory concentration (MIC) are shown in table 9 below.

TABLE 9
Antibacterial activity of inventive polypeptides in PBS buffer

| SEQ ID NO | 0.5 mM EDTA |
|---|---|
| SEQ ID NO: 132 | ≤5 |

### Example 6: Data on Antibacterial Activity of Selected Inventive Polypeptide on Various Salmonella Enteritidis Serovars in Presence of Salt

The inventors of the present invention have tested antibacterial activity of the polypeptide comprising SEQ ID NO: 132 (and further comprising a His-tag for purification reasons) on various Salmonella serovars in presence of physiological salt levels.

Briefly, exponentially growing cells of Salmonella Enteritidis were diluted 1:10 in 20 mM HEPES pH 7.4, 150 mM NaCl. Subsequently, 50 μl of cells were incubated for 1 h at 37° C. with 50 μl protein solution dissolved in 20 mM HEPES, 150 mM NaCl pH 7.4 yielding a final protein concentration of 100 μg/ml. Cells were serially diluted in 1×PBS after incubation and plated on LB agar. As a negative control, cells were incubated in the same buffer without any protein and additionally plated. Cell colonies were counted after incubation over night at 37° C. and the antibacterial activity was calculated in logarithmic units (=log 10No/Ni with N0=number of untreated colonies and Ni=number of treated colonies).

The results in form of minimal inhibitory concentration (MIC) are shown in table 10 below.

TABLE 10
Antibacterial activity of inventive polypeptides
under physiological conditions

| SEQ ID NO | Serovar | w/o EDTA |
|---|---|---|
| SEQ ID NO: 132 | Salmonella Enteritidis RKI | 3.5 |
| | 14-00409 | |
| SEQ ID NO: 132 | Salmonella Enteritidis | 4.5-5.5 |
| | LGL-246 | |
| SEQ ID NO: 132 | Salmonella Enteritidis | 6 |
| | 29/2014 | |

The polypeptide comprising SEQ ID NO: 132 thus shows significant antibacterial activity in presence of physiological salt conditions.

### Example 7: Thermostability

The inventors of the present invention have tested antibacterial activity of the polypeptide comprising SEQ ID NO: 126 on S. Typhimurium (DSM 17058) with increasing temperatures.

Briefly, prior to the determination of the MIC, the protein solution is heated to given temperatures for 20 min. Subsequently, the protein solution is stored on ice to reach a temperature of 4° C. Bacteria were grown in (Luria-Bertani) medium and diluted 1:10 in Mueller-Hinton medium. At optical density OD₆₀₀ of about 0.6 bacteria were diluted in the same medium 1:10 followed by a 1:500 dilution in the same medium. Protein solutions were pipetted into a 96 well plate, using different concentrations of proteins and an end volume of 20 μl including 500 μM EDTA final concentration. 180 μl of bacterial cells or a medium (Mueller-Hinton) control were given to the 96 well plate and mixed. The plate was incubated for 18-22 hours at 37° C. and the bacterial growth was determined measuring the OD₆₀₀ values of the wells. The MIC which is the protein concentration of the well which showed the same OD₆₀₀ value as determined for the no-bacteria control.

The results in form of minimal inhibitory concentration (MIC) are shown in table 11 below.

TABLE 11
Antibacterial activity of inventive polypeptide
with increasing temperature

| | MIC [μg/ml] |
|---|---|
| | SEQ ID NO: |
| Temp. [° C.] | 126 |
| RT | 6 |
| 70 | 12 |
| 74 | 10 |
| 78 | 10 |
| 82 | 10 |
| 86 | 10 |
| 90 | 8-10 |
| 94 | 8 |

The polypeptide comprising SEQ ID NO: 126 thus shows extreme thermostability with significant antibacterial activity even at very high temperatures.

Similar results were obtained when using the same endolysin with another peptide or when using a variant of the endolysin (e.g. SEQ ID NO:7) with such peptides.

Particularly preferred embodiments of the invention are set forth once more in items 1 to 16 below:
1. Polypeptide comprising a first and a second amino acid sequence, wherein the first amino acid sequence is an endolysin, and wherein the second amino acid sequence is an antimicrobial peptide, cationic peptide, hydrophobic peptide, amphiphatic peptide or sushi peptide, and wherein the polypeptide comprises at least one sequence selected from the following group of sequences:
   i) an amino acid sequence according to SEQ ID NO: 1;
   ii) a derivative of SEQ ID NO: 1 exhibiting at least 80% sequence identity with SEQ ID NO:1,
   iii) an amino acid sequence according to SEQ ID NO:3;
   iv) a derivative of SEQ ID NO:3 exhibiting at least 77% sequence identity with SEQ ID NO:3;
   v) an amino acid sequence according to SEQ ID NO:4; and
   vi) a derivative of SEQ ID NO:4 exhibiting at least 80% sequence identity with SEQ ID NO:4.
2. The polypeptide according to item 1, wherein the first amino acid sequence is selected from the group consisting of:
   i) an amino acid sequence according to SEQ ID NO: 1;
   ii) a derivative of SEQ ID NO: 1 exhibiting at least 80% sequence identity with SEQ ID NO:1,
   iii) an amino acid sequence according to SEQ ID NO:2; and
   iv) a derivative of SEQ ID NO:2 exhibiting at least 80% sequence identity with SEQ ID NO:2,
3. The polypeptide according to item 2, wherein the derivative of SEQ ID NO:2 exhibits at least 85%, at least 87.5%, at least 90%, at least 91%, at least 92%, at least 93%, at least 94%, at least 95%, at least 96%, at least 97%, at least 98%, at least 99%, or more than 99% sequence identity with SEQ ID NO:2.
4. The polypeptide according to item 2 or item 3, wherein said derivative of SEQ ID NO:2 comprises a sequence according to SEQ ID NO:8, in particular wherein said derivative comprises a sequence according to SEQ ID NO: 13.
5. The polypeptide according any one of items 1 to 4, wherein the derivative of SEQ ID NO:1 exhibits at least 85%, at least 87.5%, at least 90%, at least 91%, at least 92%, at least 93%, at least 94%, at least 95%, at least 96%, at least 97%, at least 98%, at least 99%, or more than 99% sequence identity with SEQ ID NO: 1.
6. The polypeptide according to anyone of the preceding items, wherein the second amino acid sequence is selected from the group consisting of:
   i) an amino acid sequence according to SEQ ID NO:3;
   ii) a derivative of SEQ ID NO:3 exhibiting at least 77% sequence identity with SEQ ID NO:3;
   iii) an amino acid sequence according to SEQ ID NO:4; and
   iv) a derivative of SEQ ID NO:4 exhibiting at least 80% sequence identity with SEQ ID NO:4.
7. The polypeptide according to item 6, wherein the derivative of SEQ ID NO:3 exhibits at least 81%, at least 86%, at least 90%, or at least 95% sequence identity with SEQ ID NO:3, or wherein the derivative of SEQ ID NO:4 exhibits at least 83%, at least 86%, at least 90%, at least 93%, or at least 96% sequence identity with SEQ ID NO:4.
8. The polypeptide according to anyone of items 1 to 7, wherein the first amino acid sequence is SEQ ID NO: 1 or said derivative thereof, and wherein the second amino acid sequence is SEQ ID NO:3 or said derivative thereof, or wherein the first amino acid sequence is SEQ ID NO:2 or said derivative thereof, and wherein the second amino acid sequence is SEQ ID NO:4 or said derivative thereof.
9. The polypeptide according to anyone of items 1 to 8, wherein said derivative of SEQ ID NO:3 is a fragment of SEQ ID NO:3, in particular a fragment according to SEQ ID NO:37.
10. The polypeptide according to item 1, wherein
   i) the first amino acid sequence is SEQ ID NO: 1 and the second amino acid sequence is SEQ ID NO:3, or
   ii) wherein the first amino acid sequence is SEQ ID NO: 13 and the second amino acid sequence is SEQ ID NO:4.
11. The polypeptide according to item 13, wherein the polypeptide comprises an amino acid sequence according to SEQ ID NO: 126, SEQ ID NO: 127, SEQ ID NO: 129 or SEQ ID NO: 130.
12. The polypeptide according to any one of items 1 to 11, wherein the polypeptide comprises an amino acid sequence as encoded by a nucleic acid sequence according to SEQ ID NO:153 or according to SEQ ID NO:154.
13. The polypeptide according to any one of the preceding items, wherein the polypeptide degrades the peptidoglycan of Salmonella bacteria.
14. Use of the polypeptide according to any one of items 1 to 13 as an antimicrobial in food, as an antimicrobial in feed, as an antimicrobial in cosmetics, or as disinfecting agent.
15. Method of controlling the growth of bacteria of the genus Salmonella in animals, in particular in livestock, companion animal and/or aquaculture, the method comprising contacting said animal, in particular the livestock, companion animal and/or aquaculture, with a polypeptide, wherein the polypeptide comprises a first and a second amino acid sequence, wherein the first amino acid sequence is an endolysin, and wherein the second amino acid sequence is an antimicrobial peptide, amphiphatic peptide, cationic peptide, hydrophobic peptide, sushi peptide or defensin, and wherein the polypeptide comprises at least one sequence selected from the following group of sequences:
   i) an amino acid sequence according to SEQ ID NO: 1;
   ii) a derivative of SEQ ID NO: 1 exhibiting at least 80% sequence identity with SEQ ID NO:1,
   iii) an amino acid sequence according to SEQ ID NO:2;
   iv) a derivative of SEQ ID NO:2 exhibiting at least 80% sequence identity with SEQ ID NO:2,
   v) an amino acid sequence according to SEQ ID NO:3;
   vi) a derivative of SEQ ID NO:3 exhibiting at least 77% sequence identity with SEQ ID NO:3;
   vii) an amino acid sequence according to SEQ ID NO:4; and
   viii) a derivative of SEQ ID NO:4 exhibiting at least 80% sequence identity with SEQ ID NO:4.
16. The method according to item 15, wherein the polypeptide is a polypeptide according to any one of items 1 to 13.

1. A polypeptide comprising a first and a second amino acid sequence, wherein the first amino acid sequence is an endolysin, and wherein the second amino acid sequence is an antimicrobial peptide, cationic peptide, hydrophobic peptide, amphiphatic peptide or sushi peptide, and wherein the polypeptide comprises at least one sequence selected from the following group of sequences:
i) an amino acid sequence according to SEQ ID NO: 1;
ii) a derivative of SEQ ID NO: 1 exhibiting at least 80% sequence identity with SEQ ID NO: 1, with the proviso that the polypeptide may not comprise the sequence of SEQ ID NO:5, if the polypeptide comprises ii), but none of iii), iv), v) or vi),
iii) an amino acid sequence according to SEQ ID NO:3;
iv) a derivative of SEQ ID NO:3 exhibiting at least 77% sequence identity with SEQ ID NO:3;
v) an amino acid sequence according to SEQ ID NO:4; with the proviso that the polypeptide may in this case not comprise the sequence of SEQ ID NO:6 in parallel; and
vi) a derivative of SEQ ID NO:4 exhibiting at least 80% sequence identity with SEQ ID NO:4.

2. The polypeptide according to claim 1, wherein the first amino acid sequence is selected from the group consisting of:
i) an amino acid sequence according to SEQ ID NO: 1;
ii) a derivative of SEQ ID NO: 1 exhibiting at least 80% sequence identity with SEQ ID NO:1,
iii) an amino acid sequence according to SEQ ID NO:2; and
iv) a derivative of SEQ ID NO:2 exhibiting at least 80% sequence identity with SEQ ID NO:2.

3. The polypeptide according to claim 2, wherein the derivative of SEQ ID NO:2 exhibits at least 85%, at least 87.5%, at least 90%, at least 91%, at least 92%, at least 93%, at least 94%, at least 95%, at least 96%, at least 97%, at least 98%, at least 99%, or more than 99% sequence identity with SEQ ID NO:2.

4. The polypeptide according to claim 2, wherein said derivative of SEQ ID NO:2 comprises a sequence according to SEQ ID NO:8, in particular wherein said derivative comprises a sequence according to SEQ ID NO: 13.

5. The polypeptide according to claim 1, wherein the derivative of SEQ ID NO:1 exhibits at least 85%, at least 87.5%, at least 90%, at least 91%, at least 92%, at least 93%, at least 94%, at least 95%, at least 96%, at least 97%, at least 98%, at least 99%, or more than 99% sequence identity with SEQ ID NO: 1.

6. The polypeptide according to claim 1, wherein the derivative of SEQ ID NO:1 exhibits at least 98%, at least 99%, or more than 99% sequence identity with SEQ ID NO: 1.

7. The polypeptide according to claim 1, wherein the derivative of SEQ ID NO: 1 is an amino acid sequence according to SEQ ID NO:7.

8. The polypeptide according to claim 1, wherein the second amino acid sequence is selected from the group consisting of:
i) an amino acid sequence according to SEQ ID NO:3;
ii) a derivative of SEQ ID NO:3 exhibiting at least 77% sequence identity with SEQ ID NO:3;
iii) an amino acid sequence according to SEQ ID NO:4; and
iv) a derivative of SEQ ID NO:4 exhibiting at least 80% sequence identity with SEQ ID NO:4.

9. The polypeptide according to claim 8, wherein the derivative of SEQ ID NO:3 exhibits at least 81%, at least 86%, at least 90%, or at least 95% sequence identity with SEQ ID NO:3, or wherein the derivative of SEQ ID NO:4 exhibits at least 83%, at least 86%, at least 90%, at least 93%, or at least 96% sequence identity with SEQ ID NO:4.

10. The polypeptide according to claim 1, wherein the polypeptide comprises an amino acid sequence according to SEQ ID NO:4; but does not comprise the amino acid sequence according to SEQ ID NO:2.

11. The polypeptide according to claim 1, wherein the first amino acid sequence is SEQ ID NO:1 or said derivative thereof, and wherein the second amino acid sequence is SEQ ID NO:3 or said derivative thereof, or wherein the first amino acid sequence is SEQ ID NO:1 or said derivative thereof, and wherein the second amino acid sequence is SEQ ID NO:4 or said derivative thereof, or wherein the first amino acid sequence is SEQ ID NO:2 or said derivative thereof, and wherein the second amino acid sequence is SEQ ID NO:3 or said derivative thereof or wherein the first amino acid sequence is SEQ ID NO:2, and wherein the second amino acid sequence is said derivative of SEQ ID NO:4, or wherein the first amino acid sequence is said derivative of SEQ ID NO:2, and wherein the second amino acid sequence is SEQ ID NO:4 or said derivative thereof.

12. The polypeptide according to claim 1, wherein said derivative of SEQ ID NO:3 is a fragment of SEQ ID NO:3, in particular a fragment according to SEQ ID NO:37.

13. The polypeptide according to claim 1, wherein
i) the first amino acid sequence is SEQ ID NO: 1 and the second amino acid sequence is SEQ ID NO:3,
ii) the first amino acid sequence is SEQ ID NO: 1 and the second amino acid sequence is SEQ ID NO:4,
iii) the first amino acid sequence is SEQ ID NO: 13 and the second amino acid sequence is SEQ ID NO:3, or
iii) wherein the first amino acid sequence is SEQ ID NO:13 and the second amino acid sequence is SEQ ID NO:4.

14. The polypeptide according to claim 13, wherein the polypeptide comprises an amino acid sequence according to SEQ ID NO: 126, SEQ ID NO: 127, SEQ ID NO: 129, SEQ ID NO: 130, SEQ ID NO: 132, SEQ ID NO: 133, SEQ ID NO: 135 or SEQ ID NO: 136.

15. The polypeptide according to claim 1, wherein the polypeptide comprises an amino acid sequence as encoded by a nucleic acid sequence according to SEQ ID NO:153 or according to SEQ ID NO:154.

16. The polypeptide according to claim 1, wherein the polypeptide degrades the peptidoglycan of Salmonella bacteria.

17. A nucleic acid encoding a polypeptide according to claim 1.

18. A vector comprising a nucleic acid according to claim 17.

19. A host cell comprising a nucleic acid according to claim 17.

20. A composition comprising a polypeptide according to claim 1.

21. The composition according to claim 20, wherein the composition is a pharmaceutical composition comprising a pharmaceutical acceptable diluent, excipient or carrier.

22. (canceled)

23. A method of treating or preventing infections caused by bacteria of the genus Salmonella comprising administering to a subject in need thereof a polypeptide, a nucleic acid encoding said polypeptide, or a vector comprising said nucleic acid encoding said polypeptide, wherein the polypeptide comprises a first and a second amino acid sequence, wherein the first amino acid sequence is an endolysin, and wherein the second amino acid sequence is an antimicrobial peptide, amphiphatic peptide, cationic peptide, hydrophobic peptide, sushi peptide or defensin, and wherein the polypeptide comprises at least one sequence selected from the following group of sequences:
i) an amino acid sequence according to SEQ ID NO: 1;
ii) a derivative of SEQ ID NO:1 exhibiting at least 80% sequence identity with SEQ ID NO:1,
iii) an amino acid sequence according to SEQ ID NO:2;
iv) a derivative of SEQ ID NO:2 exhibiting at least 80% sequence identity with SEQ ID NO:2,
v) an amino acid sequence according to SEQ ID NO:3;
vi) a derivative of SEQ ID NO:3 exhibiting at least 77% sequence identity with SEQ ID NO:3;
vii) an amino acid sequence according to SEQ ID NO:4; and
viii) a derivative of SEQ ID NO:4 exhibiting at least 80% sequence identity with SEQ ID NO:4.

24-25. (canceled)

26. A method of controlling the growth of bacteria of the genus Salmonella in animals, in particular in livestock, companion animal and/or aquaculture, the method comprising contacting said animal, in particular the livestock, companion animal and/or aquaculture, with a polypeptide, a nucleic acid encoding such polypeptide, a vector comprising such nucleic acid, a host cell comprising such polypeptide, nucleic acid and/or vector, or a composition comprising such polypeptide, nucleic acid, vector, and/or host cell, wherein the polypeptide comprises a first and a second amino acid sequence, wherein the first amino acid sequence is an endolysin, and wherein the second amino acid sequence is an antimicrobial peptide, amphiphatic peptide, cationic peptide, hydrophobic peptide, sushi peptide or defensin, and wherein the polypeptide comprises at least one sequence selected from the following group of sequences:
i) an amino acid sequence according to SEQ ID NO: 1;
ii) a derivative of SEQ ID NO: 1 exhibiting at least 80% sequence identity with SEQ ID NO:1,
iii) an amino acid sequence according to SEQ ID NO:2;
iv) a derivative of SEQ ID NO:2 exhibiting at least 80% sequence identity with SEQ ID NO:2,
v) an amino acid sequence according to SEQ ID NO:3;
vi) a derivative of SEQ ID NO:3 exhibiting at least 77% sequence identity with SEQ ID NO:3;
vii) an amino acid sequence according to SEQ ID NO:4; and
viii) a derivative of SEQ ID NO:4 exhibiting at least 80% sequence identity with SEQ ID NO:4.

27. The method according to claim 26, wherein the polypeptide is a polypeptide according to claim 1.


SEQUENCE LISTING (154 sequences)

SEQ ID NO: 1 - protein, 136 aa, Unknown
  S394 endolysin without N-terminal methionine
SFKFGKNSEKQLATVKPELQKVARRALELSPYDFTIVQGIRTVAQSAQNIANGTSFLKDPSKSKHVTGDAIDFAPYINGKIDWKDLEAFWAVKKAFEQAGKELGIKLRFGADWNSSGDYHDEIDRGTYDGGHVELV

SEQ ID NO: 2 - protein, 259 aa, unknown
  KZ144 endolysin without N-terminal methionine
KVLRKGDRGDEVCQLQTLLNLCGYDVGKPDGIFGNNTFNQVVKFQKDNCLDSDGIVGKNTWAELFSKYSPPIPYKTIPMPTANKSRAAATPVMNAVENATGVRSQLLLTFASIESAFDYEIKAKTSSATGWFQFLTGTWKTMIENYGMKYGVLTDPTGALRKDPRISALMGAELIKENMNILRPVLKREPTDTDLYLAHFFGPGAARRFLTTGQNELAATHFPKEAQANPSIFYNKDGSPKTIQEVYNLMDGKVAAHRK

SEQ ID NO: 3 - protein, 22 aa, Artificial sequence
  MSI-78 peptide
GIGKFLKKAKKFGKAFVKILKK

SEQ ID NO: 4 - protein, 30 aa, Bungarus fasciatus
  misc_feature (1)..(30): Cathelicidin-BF peptide
KFFRKLKKSVKKRAKEFFKKPRVIGVSIPF

SEQ ID NO: 5 - protein, 137 aa, Escherichia virus T5
MSFKFGKNSEKQLATVKPELQKVARRALELSPYDFTIVQGIRTVAQSAQNIANGTSFLKDPSKSKHITGDAIDFAPYINGKIDWNDLEAFWAVKKAFEQAGKELGIKLRFGADWNASGDYHDEIKRGTYDGGHVELV

SEQ ID NO: 6 - protein, 269 aa, artificial
  GS linker plus KZ144 endolysin w/o methionine      plus His tag
GSKVLRKGDRGDEVCQLQTLLNLCGYDVGKPDGIFGNNTFNQVVKFQKDNCLDSDGIVGKNTWAELFSKYSPPIPYKTIPMPTANKSRAAATPVMNAVENATGVRSQLLLTFASIESAFDYEIKAKTSSATGWFQFLTGTWKTMIENYGMKYGVLTDPTGALRKDPRISALMGAELIKENMNILRPVLKREPTDTDLYLAHFFGPGAARRFLTTGQNELAATHFPKEAQANPSIFYNKDGSPKTIQEVYNLMDGKVAAHRKLEHHHHHH

SEQ ID NO: 7 - protein, 136 aa, Escherichia virus T5
SFKFGKNSEKQLATVKPELQKVARRALELSPYDFTIVQGIRTVAQSAQNIANGTSFLKDPSKSKHITGDAIDFAPYINGKIDWNDLEAFWAVKKAFEQAGKELGIKLRFGADWNASGDYHDEIKRGTYDGGHVELV

SEQ ID NO: 8 - protein, 260 aa, Artificial
  Consensus Sequence
  MISC_FEATURE (1)..(1): Xaa can be any naturally occurring amino acid      or absent; in particular it can be methionine
  MISC_FEATURE (14)..(14): Xaa can be any naturally occurring amino acid,      in particular serine, arginine or aspargine
  MISC_FEATURE (23)..(23): Xaa can be any naturally occurring amino acid,      in particular serine, arginine or aspargine
  MISC_FEATURE (50)..(50): Xaa can be any naturally occurring amino acid,      in particular serine, arginine or aspargine
  MISC_FEATURE (82)..(82): Xaa can be any naturally occurring amino acid,      in particular threonine or isoleucine
  MISC_FEATURE (122)..(122): Xaa can be any naturally occurring amino acid,      in particular isoleucine or methionine
  MISC_FEATURE (149)..(149): Xaa can be any naturally occurring amino acid,      in particular methionine or proline
  MISC_FEATURE (154)..(154): Xaa can be any naturally occurring amino acid,      in particular leucine or threonine
  MISC_FEATURE (160)..(160): Xaa can be any naturally occurring amino acid,      in particular alanine or threonine
  MISC_FEATURE (167)..(167): Xaa can be any naturally occurring amino acid,      in particular isoleucine or leucine
  MISC_FEATURE (179)..(179): Xaa can be any naturally occurring amino acid,      in particular asparagine or phenylalanine
  MISC_FEATURE (180)..(180): Xaa can be any naturally occurring amino acid,      in particular methionine or glutamic acid
  MISC_FEATURE (186)..(186): Xaa can be any naturally occurring amino acid,      in particular valine or tyrosine
  MISC_FEATURE (206)..(206): Xaa can be any naturally occurring amino acid,      in particular alanine, asparagine or valine
  MISC_FEATURE (212)..(212): Xaa can be any naturally occurring amino acid,      in particular threonine or asparagine
  MISC_FEATURE (224)..(224): Xaa can be any naturally occurring amino acid,      in particular proline or glutamine
  MISC_FEATURE (230)..(230): Xaa can be any naturally occurring amino acid,      in particular asparagine or tyrosine
  MISC_FEATURE (232)..(232): Xaa can be any naturally occurring amino acid,      in particular serine or threonine
XKVLRKGDRGDEVXQLQTLLNLXGYDVGKPDGIFGNNTFNQVVKFQKDNXLDSDGIVGKNTWAELFSKYSPPIPYKTIPMPXANKSRAAATPVMNAVENATGVRSQLLLTFASIESAFDYEXKAKTSSATGWFQFLTGTWKTMIENYGXKYGVXTDPTGXLRKDPRXSALMGAELIKEXXNILRPXLKREPTDTDLYLAHFFGPGXARRFLXTGQNELAATHFXKEAQAXPXIFYNKDGSPKTIQEVYNLMDGKVAAHRK

SEQ ID NO: 9 - protein, 259 aa, artificial sequence
  mutated KZ144 with C14S and C50S, without      N-terminal methionine
KVLRKGDRGDEVSQLQTLLNLCGYDVGKPDGIFGNNTFNQVVKFQKDNSLDSDGIVGKNTWAELFSKYSPPIPYKTIPMPTANKSRAAATPVMNAVENATGVRSQLLLTFASIESAFDYEIKAKTSSATGWFQFLTGTWKTMIENYGMKYGVLTDPTGALRKDPRISALMGAELIKENMNILRPVLKREPTDTDLYLAHFFGPGAARRFLTTGQNELAATHFPKEAQANPSIFYNKDGSPKTIQEVYNLMDGKVAAHRK

SEQ ID NO: 10 - protein, 259 aa, artificial sequence
  mutated KZ144 with T82I, A206V and S232,      without N-terminal methionine
KVLRKGDRGDEVCQLQTLLNLCGYDVGKPDGIFGNNTFNQVVKFQKDNCLDSDGIVGKNTWAELFSKYSPPIPYKTIPMPIANKSRAAATPVMNAVENATGVRSQLLLTFASIESAFDYEIKAKTSSATGWFQFLTGTWKTMIENYGMKYGVLTDPTGALRKDPRISALMGAELIKENMNILRPVLKREPTDTDLYLAHFFGPGVARRFLTTGQNELAATHFPKEAQANPTIFYNKDGSPKTIQEVYNLMDGKVAAHRK

SEQ ID NO: 11 - protein, 259 aa, artificial sequence
  mutated KZ144 with T82I, A206V, S232T, I122M      and A160T, without N-terminal methionine
KVLRKGDRGDEVCQLQTLLNLCGYDVGKPDGIFGNNTFNQVVKFQKDNCLDSDGIVGKNTWAELFSKYSPPIPYKTIPMPIANKSRAAATPVMNAVENATGVRSQLLLTFASIESAFDYEMKAKTSSATGWFQFLTGTWKTMIENYGMKYGVLTDPTGTLRKDPRISALMGAELIKENMNILRPVLKREPTDTDLYLAHFFGPGVARRFLTTGQNELAATHFPKEAQANPTIFYNKDGSPKTIQEVYNLMDGKVAAHRK

SEQ ID NO: 12 - protein, 259 aa, artificial sequence
  mutated KZ144 with C14S, C50S, I122M and A160T,      without N-terminal methionine
KVLRKGDRGDEVSQLQTLLNLCGYDVGKPDGIFGNNTFNQVVKFQKDNSLDSDGIVGKNTWAELFSKYSPPIPYKTIPMPTANKSRAAATPVMNAVENATGVRSQLLLTFASIESAFDYEMKAKTSSATGWFQFLTGTWKTMIENYGMKYGVLTDPTGTLRKDPRISALMGAELIKENMNILRPVLKREPTDTDLYLAHFFGPGAARRFLTTGQNELAATHFPKEAQANPSIFYNKDGSPKTIQEVYNLMDGKVAAHRK

SEQ ID NO: 13 - protein, 259 aa, artificial sequence
  mutated KZ144 with C14S, C23S and C50S, without      N-terminal methionine
KVLRKGDRGDEVSQLQTLLNLSGYDVGKPDGIFGNNTFNQVVKFQKDNSLDSDGIVGKNTWAELFSKYSPPIPYKTIPMPTANKSRAAATPVMNAVENATGVRSQLLLTFASIESAFDYEIKAKTSSATGWFQFLTGTWKTMIENYGMKYGVLTDPTGALRKDPRISALMGAELIKENMNILRPVLKREPTDTDLYLAHFFGPGAARRFLTTGQNELAATHFPKEAQANPSIFYNKDGSPKTIQEVYNLMDGKVAAHRK

SEQ ID NO: 14 - protein, 259 aa, artificial sequence
  mutated KZ144 with T82I, A206V, S232T, I122M,      A160T, C14S and C50S, without N-terminal methionine
KVLRKGDRGDEVSQLQTLLNLCGYDVGKPDGIFGNNTFNQVVKFQKDNSLDSDGIVGKNTWAELFSKYSPPIPYKTIPMPIANKSRAAATPVMNAVENATGVRSQLLLTFASIESAFDYEMKAKTSSATGWFQFLTGTWKTMIENYGMKYGVLTDPTGTLRKDPRISALMGAELIKENMNILRPVLKREPTDTDLYLAHFFGPGVARRFLTTGQNELAATHFPKEAQANPTIFYNKDGSPKTIQEVYNLMDGKVAAHRK

SEQ ID NO: 15 - protein, 259 aa, artificial sequence
  mutated KZ144 with T82I, A206N, S232T, I122M,      A160T, C14S and  C50S, without N-terminal methionine
KVLRKGDRGDEVSQLQTLLNLCGYDVGKPDGIFGNNTFNQVVKFQKDNSLDSDGIVGKNTWAELFSKYSPPIPYKTIPMPIANKSRAAATPVMNAVENATGVRSQLLLTFASIESAFDYEMKAKTSSATGWFQFLTGTWKTMIENYGMKYGVLTDPTGTLRKDPRISALMGAELIKENMNILRPVLKREPTDTDLYLAHFFGPGNARRFLTTGQNELAATHFPKEAQANPTIFYNKDGSPKTIQEVYNLMDGKVAAHRK

SEQ ID NO: 16 - protein, 259 aa, Artificial Sequence
  mutated KZ144 with N230Y, T82I, A206V, S232T,      I122M, A160T, C14S and C50S, without N-terminal methionine
KVLRKGDRGDEVSQLQTLLNLCGYDVGKPDGIFGNNTFNQVVKFQKDNSLDSDGIVGKNTWAELFSKYSPPIPYKTIPMPIANKSRAAATPVMNAVENATGVRSQLLLTFASIESAFDYEMKAKTSSATGWFQFLTGTWKTMIENYGMKYGVLTDPTGTLRKDPRISALMGAELIKENMNILRPVLKREPTDTDLYLAHFFGPGVARRFLTTGQNELAATHFPKEAQAYPTIFYNKDGSPKTIQEVYNLMDGKVAAHRK

SEQ ID NO: 17 - protein, 259 aa, Artificial Sequence
  mutated KZ144 with M180E, T82I, A206V, S232T,      I122M, A160T, C14S and C50S, without N-terminal methionine
KVLRKGDRGDEVSQLQTLLNLCGYDVGKPDGIFGNNTFNQVVKFQKDNSLDSDGIVGKNTWAELFSKYSPPIPYKTIPMPIANKSRAAATPVMNAVENATGVRSQLLLTFASIESAFDYEMKAKTSSATGWFQFLTGTWKTMIENYGMKYGVLTDPTGTLRKDPRISALMGAELIKENENILRPVLKREPTDTDLYLAHFFGPGVARRFLTTGQNELAATHFPKEAQANPTIFYNKDGSPKTIQEVYNLMDGKVAAHRK

SEQ ID NO: 18 - protein, 259 aa, Artificial Sequence
  mutated KZ144 with M149P, T82I, A206V, S232T,      I122M, A160T, C14S and C50S, without N-terminal methionine
KVLRKGDRGDEVSQLQTLLNLCGYDVGKPDGIFGNNTFNQVVKFQKDNSLDSDGIVGKNTWAELFSKYSPPIPYKTIPMPIANKSRAAATPVMNAVENATGVRSQLLLTFASIESAFDYEMKAKTSSATGWFQFLTGTWKTMIENYGPKYGVLTDPTGTLRKDPRISALMGAELIKENMNILRPVLKREPTDTDLYLAHFFGPGVARRFLTTGQNELAATHFPKEAQANPTIFYNKDGSPKTIQEVYNLMDGKVAAHRK

SEQ ID NO: 19 - protein, 259 aa, Artificial Sequence
  mutated KZ144 with V186Y, T82I, A206V, S232T,      I122M, A160T, C14S and C50S, without N-terminal methionine
KVLRKGDRGDEVSQLQTLLNLCGYDVGKPDGIFGNNTFNQVVKFQKDNSLDSDGIVGKNTWAELFSKYSPPIPYKTIPMPIANKSRAAATPVMNAVENATGVRSQLLLTFASIESAFDYEMKAKTSSATGWFQFLTGTWKTMIENYGMKYGVLTDPTGTLRKDPRISALMGAELIKENMNILRPYLKREPTDTDLYLAHFFGPGVARRFLTTGQNELAATHFPKEAQANPTIFYNKDGSPKTIQEVYNLMDGKVAAHRK

SEQ ID NO: 20 - protein, 259 aa, Artificial Sequence
  mutated KZ144 with T82I, A206V, S232T, I122M,      A160T, C14R and C50S, without N-terminal methionine
KVLRKGDRGDEVRQLQTLLNLCGYDVGKPDGIFGNNTFNQVVKFQKDNSLDSDGIVGKNTWAELFSKYSPPIPYKTIPMPIANKSRAAATPVMNAVENATGVRSQLLLTFASIESAFDYEMKAKTSSATGWFQFLTGTWKTMIENYGMKYGVLTDPTGTLRKDPRISALMGAELIKENMNILRPVLKREPTDTDLYLAHFFGPGVARRFLTTGQNELAATHFPKEAQANPTIFYNKDGSPKTIQEVYNLMDGKVAAHRK

SEQ ID NO: 21 - protein, 259 aa, Artificial Sequence
  mutated KZ144 with T82I, A206V, S232T, I122M,      C14S and C50S, without N-terminal methionine
KVLRKGDRGDEVCQLQTLLNLCGYDVGKPDGIFGNNTFNQVVKFQKDNSLDSDGIVGKNTWAELFSKYSPPIPYKTIPMPIANKSRAAATPVMNAVENATGVRSQLLLTFASIESAFDYEMKAKTSSATGWFQFLTGTWKTMIENYGMKYGVLTDPTGALRKDPRISALMGAELIKENMNILRPVLKREPTDTDLYLAHFFGPGVARRFLTTGQNELAATHFPKEAQANPTIFYNKDGSPKTIQEVYNLMDGKVAAHRK

SEQ ID NO: 22 - protein, 259 aa, Artificial Sequence
  mutated KZ144 with T82I, A206V, S232T, I122M,      A160T, C14S and C50N, without N-terminal methionine
KVLRKGDRGDEVSQLQTLLNLCGYDVGKPDGIFGNNTFNQVVKFQKDNNLDSDGIVGKNTWAELFSKYSPPIPYKTIPMPIANKSRAAATPVMNAVENATGVRSQLLLTFASIESAFDYEMKAKTSSATGWFQFLTGTWKTMIENYGMKYGVLTDPTGTLRKDPRISALMGAELIKENMNILRPVLKREPTDTDLYLAHFFGPGVARRFLTTGQNELAATHFPKEAQANPTIFYNKDGSPKTIQEVYNLMDGKVAAHRK

SEQ ID NO: 23 - protein, 259 aa, Artificial Sequence
  mutated KZ144 with C14R, C50S, T82I, I122M,      M149P, A206V and S232T; without N-terminal methionine
KVLRKGDRGDEVRQLQTLLNLCGYDVGKPDGIFGNNTFNQVVKFQKDNSLDSDGIVGKNTWAELFSKYSPPIPYKTIPMPIANKSRAAATPVMNAVENATGVRSQLLLTFASIESAFDYEMKAKTSSATGWFQFLTGTWKTMIENYGPKYGVLTDPTGALRKDPRISALMGAELIKENMNILRPVLKREPTDTDLYLAHFFGPGVARRFLTTGQNELAATHFPKEAQANPTIFYNKDGSPKTIQEVYNLMDGKVAAHRK

SEQ ID NO: 24 - protein, 259 aa, Artificial Sequence
  mutated KZ144 with C14R, C50S, T82I, I122M,      M149P, A160T, A206V and S232T; without N-terminal methionine
KVLRKGDRGDEVRQLQTLLNLCGYDVGKPDGIFGNNTFNQVVKFQKDNSLDSDGIVGKNTWAELFSKYSPPIPYKTIPMPIANKSRAAATPVMNAVENATGVRSQLLLTFASIESAFDYEMKAKTSSATGWFQFLTGTWKTMIENYGPKYGVLTDPTGTLRKDPRISALMGAELIKENMNILRPVLKREPTDTDLYLAHFFGPGVARRFLTTGQNELAATHFPKEAQANPTIFYNKDGSPKTIQEVYNLMDGKVAAHRK

SEQ ID NO: 25 - protein, 259 aa, Artificial Sequence
  mutated KZ144 with T82I, I122M, M149P, A206V,      S232T, C14R and C50N, without N-terminal methionine
KVLRKGDRGDEVRQLQTLLNLCGYDVGKPDGIFGNNTFNQVVKFQKDNNLDSDGIVGKNTWAELFSKYSPPIPYKTIPMPIANKSRAAATPVMNAVENATGVRSQLLLTFASIESAFDYEMKAKTSSATGWFQFLTGTWKTMIENYGPKYGVLTDPTGALRKDPRISALMGAELIKENMNILRPVLKREPTDTDLYLAHFFGPGVARRFLTTGQNELAATHFPKEAQANPTIFYNKDGSPKTIQEVYNLMDGKVAAHRK

SEQ ID NO: 26 - protein, 259 aa, Artificial Sequence
  mutated KZ144 with T82I, I122M, M149P, I167L,      A206V, S232T, C14R and C50N, without N-terminal methionine
KVLRKGDRGDEVRQLQTLLNLCGYDVGKPDGIFGNNTFNQVVKFQKDNNLDSDGIVGKNTWAELFSKYSPPIPYKTIPMPIANKSRAAATPVMNAVENATGVRSQLLLTFASIESAFDYEMKAKTSSATGWFQFLTGTWKTMIENYGPKYGVLTDPTGALRKDPRLSALMGAELIKENMNILRPVLKREPTDTDLYLAHFFGPGVARRFLTTGQNELAATHFPKEAQANPTIFYNKDGSPKTIQEVYNLMDGKVAAHRK

SEQ ID NO: 27 - protein, 259 aa, Artificial Sequence
  mutated KZ144 with T82I, I122M, M149P,  N179F,      A206V, S232T, C14R and C50N, without N-terminal methionine
KVLRKGDRGDEVRQLQTLLNLCGYDVGKPDGIFGNNTFNQVVKFQKDNNLDSDGIVGKNTWAELFSKYSPPIPYKTIPMPIANKSRAAATPVMNAVENATGVRSQLLLTFASIESAFDYEMKAKTSSATGWFQFLTGTWKTMIENYGPKYGVLTDPTGALRKDPRLSALMGAELIKEFMNILRPVLKREPTDTDLYLAHFFGPGVARRFLTTGQNELAATHFPKEAQANPTIFYNKDGSPKTIQEVYNLMDGKVAAHRK

SEQ ID NO: 28 - protein, 259 aa, Artificial Sequence
  mutated KZ144 with T82I, I122M, M149P,  A206V,      T212N, S232T, C14R and C50N, without N-terminal methionine
KVLRKGDRGDEVRQLQTLLNLCGYDVGKPDGIFGNNTFNQVVKFQKDNNLDSDGIVGKNTWAELFSKYSPPIPYKTIPMPIANKSRAAATPVMNAVENATGVRSQLLLTFASIESAFDYEMKAKTSSATGWFQFLTGTWKTMIENYGPKYGVLTDPTGALRKDPRISALMGAELIKENMNILRPVLKREPTDTDLYLAHFFGPGVARRFLNTGQNELAATHFPKEAQANPTIFYNKDGSPKTIQEVYNLMDGKVAAHRK

SEQ ID NO: 29 - protein, 259 aa, Artificial Sequence
  mutated KZ144 with T82I, I122M, M149P,  A206V,      P224Q, S232T, C14R and C50N, without N-terminal methionine
KVLRKGDRGDEVRQLQTLLNLCGYDVGKPDGIFGNNTFNQVVKFQKDNNLDSDGIVGKNTWAELFSKYSPPIPYKTIPMPIANKSRAAATPVMNAVENATGVRSQLLLTFASIESAFDYEMKAKTSSATGWFQFLTGTWKTMIENYGPKYGVLTDPTGALRKDPRISALMGAELIKENMNILRPVLKREPTDTDLYLAHFFGPGVARRFLTTGQNELAATHFQKEAQANPTIFYNKDGSPKTIQEVYNLMDGKVAAHRK

SEQ ID NO: 30 - protein, 259 aa, Artificial Sequence
  mutated KZ144 with T82I, I122M, M149P, L154T,      A206V, S232T, C14R and C50N, without N-terminal methionine
KVLRKGDRGDEVRQLQTLLNLCGYDVGKPDGIFGNNTFNQVVKFQKDNNLDSDGIVGKNTWAELFSKYSPPIPYKTIPMPIANKSRAAATPVMNAVENATGVRSQLLLTFASIESAFDYEMKAKTSSATGWFQFLTGTWKTMIENYGPKYGVTTDPTGALRKDPRISALMGAELIKENMNILRPVLKREPTDTDLYLAHFFGPGVARRFLTTGQNELAATHFPKEAQANPTIFYNKDGSPKTIQEVYNLMDGKVAAHRK

SEQ ID NO: 31 - protein, 137 aa, Bacteriophage S-394
MSFKFGKNSEKQLATVKPELQKVARRALELSPYDFTIVQGIRTVAQSAQNIANGTSFLKDPSKSKHVTGDAIDFAPYINGKIDWKDLEAFWAVKKAFEQAGKELGIKLRFGADWNSSGDYHDEIDRGTYDGGHVELV

SEQ ID NO: 32 - protein, 260 aa, unknown
  phiKZgp144
MKVLRKGDRGDEVCQLQTLLNLCGYDVGKPDGIFGNNTFNQVVKFQKDNCLDSDGIVGKNTWAELFSKYSPPIPYKTIPMPTANKSRAAATPVMNAVENATGVRSQLLLTFASIESAFDYEIKAKTSSATGWFQFLTGTWKTMIENYGMKYGVLTDPTGALRKDPRISALMGAELIKENMNILRPVLKREPTDTDLYLAHFFGPGAARRFLTTGQNELAATHFPKEAQANPSIFYNKDGSPKTIQEVYNLMDGKVAAHRK

SEQ ID NO: 33 - protein, 260 aa, artificial sequence
  mutated KZ144 with C14S, C23S and C50S
MKVLRKGDRGDEVSQLQTLLNLSGYDVGKPDGIFGNNTFNQVVKFQKDNSLDSDGIVGKNTWAELFSKYSPPIPYKTIPMPTANKSRAAATPVMNAVENATGVRSQLLLTFASIESAFDYEIKAKTSSATGWFQFLTGTWKTMIENYGMKYGVLTDPTGALRKDPRISALMGAELIKENMNILRPVLKREPTDTDLYLAHFFGPGAARRFLTTGQNELAATHFPKEAQANPSIFYNKDGSPKTIQEVYNLMDGKVAAHRK

SEQ ID NO: 34 - protein, 327 aa, unknown
  OBPgp279
KNSEKNASIIMSIQRTLASLSLYGGRIDGLFGEKCRGAIILMLNKVYPNFSTNKLPSNTYEAESVFTFLQTALAGVGLYTITIDGKWGGTSQGAIDALVKSYRQITEAERAGSTLPLGLATVMSKHMSIEQLRAMLPTDRQGYAEVYIDPLNETMDIFEINTPLRIAHFMAQILHETACFKYTEELASGKAYEGRADLGNTRPGDGPLFKGRGLLQITGRLNYVKCQVYLREKLKDPTFDITSSVTCAQQLSESPLLAALASGYFWRFIKPKLNETADKDDIYWVSVYVNGYAKQANPYYPNRDKEPNHMKERVQMLAVTKKALGIV

SEQ ID NO: 35 - protein, 235 aa, unknown
  PVP-SE1gp146
NAAIAEIQRMLIEGGFSVGKSGADGLYGPATKAALQKCIAQATSGNNKGGTLKLTQAQLDKIFPVGASSGRNAKFLKPLNDLFEKTEINTVNRVAGFLSQIGVESAEFRYVRELGNDAYFDKYDTGPIAERLGNTPQKDGDGAKYKGRGLIQVTGLANYKACGKALGLDLVNHPELLEQPEYAVASAGWYWDTRNINAACDADDIVKITKLVNGGTNHLAERTAYYKKAKSVLTS

SEQ ID NO: 36 - protein, 300 aa, artificial
  SEQ ID NO 124 of WO2015/155244
MKFFRKLKKSVKKRAKEFFKKPRVIGVSIPFGSKVLRKGDRGDEVCQLQTLLNLCGYDVGKPDGIFGNNTFNQVVKFQKDNCLDSDGIVGKNTWAELFSKYSPPIPYKTIPMPTANKSRAAATPVMNAVENATGVRSQLLLTFASIESAFDYEIKAKTSSATGWFQFLTGTWKTMIENYGMKYGVLTDPTGALRKDPRISALMGAELIKENMNILRPVLKREPTDTDLYLAHFFGPGAARRFLTTGQNELAATHFPKEAQANPSIFYNKDGSPKTIQEVYNLMDGKVAAHRKLEHHHHHH

SEQ ID NO: 37 - protein, 17 aa, Artificial Sequence
  MSI-78 (4-20)  peptide
KFLKKAKKFGKAFVKIL

SEQ ID NO: 38 - protein, 6 aa, artificial
  synthetic sequence
KRKKRK

SEQ ID NO: 39 - protein, 5 aa, artificial
  synethtic sequence
  misc_feature (3)..(3): Xaa can be any naturally occurring amino acid
KRXKR

SEQ ID NO: 40 - protein, 5 aa, artificial
  synthetic sequence
KRSKR

SEQ ID NO: 41 - protein, 5 aa, artificial
  synthetic sequence
KRGSG

SEQ ID NO: 42 - protein, 9 aa, artificial
  synthetic sequence
KRKKRKKRK

SEQ ID NO: 43 - protein, 9 aa, artificial
  synthetic sequence
RRRRRRRRR

SEQ ID NO: 44 - protein, 8 aa, artificial
  synthetic sequence
KKKKKKKK

SEQ ID NO: 45 - protein, 10 aa, artificial
  synthetic sequence
KRKKRKKRKK

SEQ ID NO: 46 - protein, 12 aa, artificial
  synthetic sequence
KRKKRKKRKKRK

SEQ ID NO: 47 - protein, 14 aa, artificial
  synthetic sequence
KRKKRKKRKKRKKR

SEQ ID NO: 48 - protein, 16 aa, artificial
  synthetic sequence
KKKKKKKKKKKKKKKK

SEQ ID NO: 49 - protein, 18 aa, artificial
  synthetic sequence
KRKKRKKRKKRKKRKKRK

SEQ ID NO: 50 - protein, 19 aa, artificial
  synthetic sequence
KRKKRKKRKKRKKRKKRKK

SEQ ID NO: 51 - protein, 19 aa, artificial
  synthetic sequence
RRRRRRRRRRRRRRRRRRR

SEQ ID NO: 52 - protein, 19 aa, artificial
  synthetic sequence
KKKKKKKKKKKKKKKKKKK

SEQ ID NO: 53 - protein, 20 aa, artificial
  synthetic sequence
KRKKRKKRKRSKRKKRKKRK

SEQ ID NO: 54 - protein, 21 aa, artificial
  synthetic sequence
KRKKRKKRKRSKRKKRKKRKK

SEQ ID NO: 55 - protein, 21 aa, artificial
  synthetic sequence
KRKKRKKRKKRKKRKKRKKRK

SEQ ID NO: 56 - protein, 22 aa, artificial
  synthetic sequence
KRKKRKKRKRGSGKRKKRKKRK

SEQ ID NO: 57 - protein, 24 aa, artificial
  synthetic sequence
KRKKRKKRKRGSGSGKRKKRKKRK

SEQ ID NO: 58 - protein, 25 aa, artificial
  synthetic sequence
KRKKRKKRKKRKKRKKRKKRKKRKK

SEQ ID NO: 59 - protein, 31 aa, artificial
  synthetic sequence
KRKKRKKRKRSKRKKRKKRKRSKRKKRKKRK

SEQ ID NO: 60 - protein, 38 aa, artificial
  synthetic sequence
KRKKRKKRKRGSGSGKRKKRKKRKGSGSGKRKKRKKRK

SEQ ID NO: 61 - protein, 39 aa, artificial
  synthetic sequence
KRKKRKKRKKRKKRKKRKKRKKRKKRKKRKKRKKRKKRK

SEQ ID NO: 62 - protein, 42 aa, artificial
  synthetic sequence
KRKKRKKRKRSKRKKRKKRKRSKRKKRKKRKRSKRKKRKKRK

SEQ ID NO: 63 - protein, 37 aa, Homo sapiens
LLGDFFRKSKEKIGKEFKRIVQRIKDFLRNLVPRTES

SEQ ID NO: 64 - protein, 29 aa, unknown
  SMAP-29 sheep
RGLRRLGRKIAHGVKKYGPTVLRIIRIAG

SEQ ID NO: 65 - protein, 13 aa, unknown
  Indolicidine bovine
ILPWKWPWWPWRR

SEQ ID NO: 66 - protein, 18 aa, unknown
  Protegrin Porcine
RGGRLCYCRRRFCVCVGR

SEQ ID NO: 67 - protein, 31 aa, unknown
  Cecropin P1 Mammal (pig)
SWLSKTAKKLENSAKKRISEGIAIAIQGGPR

SEQ ID NO: 68 - protein, 23 aa, unknown
  Magainin frog
GIGKFLHSAKKFGKAFVGEIMNS

SEQ ID NO: 69 - protein, 25 aa, unknown
  Pleurocidin fish
GWGSFFKKAAHVGKHVGKAALTHYL

SEQ ID NO: 70 - protein, 36 aa, Aedes aegypti
GGLKKLGKKLEGAGKRVFNAAEKALPVVAGAKALRK

SEQ ID NO: 71 - protein, 40 aa, Drosophila melanogaster
GWLKKIGKKIERVGQHTRDATIQGLGIPQQAANVAATARG

SEQ ID NO: 72 - protein, 21 aa, unknown
  Buforin II vertebrate
TRSSRAGLQFPVGRVHRLLRK

SEQ ID NO: 73 - protein, 39 aa, unknown
  Sarcotoxin IA Fly
GWLKKIGKKIERVGQHTRDATIQGLGIAQQAANVAATAR

SEQ ID NO: 74 - protein, 17 aa, Apis mellifera
ANRPVYIPPPRPPHPRL

SEQ ID NO: 75 - protein, 24 aa, unknown
  Ascaphine 5 Frog
GIKDWIKGAAKKLIKTVASHIANQ

SEQ ID NO: 76 - protein, 22 aa, unknown
  Nigrocine 2 Frog
GLLSKVLGVGKKVLCGVSGLVC

SEQ ID NO: 77 - protein, 24 aa, unknown
  Pseudin 1 Rana Frog
GLNTLKKVFQGLHEAIKLINNHVQ

SEQ ID NO: 78 - protein, 18 aa, unknown
  Ranalexin Frog
FLGGLIVPAMICAVTKKC

SEQ ID NO: 79 - protein, 26 aa, unknown
  Melittin bee
GIGAVLKVLTTGLPALISWIKRKRQQ

SEQ ID NO: 80 - protein, 25 aa, unknown
  Lycotoxin 1 Spider
IWLTALKFLGKHAAKKLAKQQLSKL

SEQ ID NO: 81 - protein, 19 aa, unknown
  Parasin 1 Fish
KGRGKQGGKVRAKAKTRSS

SEQ ID NO: 82 - protein, 39 aa, unknown
  Buforin I Toad
AGRGKQGGKVRAKAKTRSSRAGLQFPVGRVHRLLRKGNY

SEQ ID NO: 83 - protein, 34 aa, unknown
  Dermaseptin 1 Frog
ALWKTMLKKLGTMALHAGKAALGAAADTISQGTQ

SEQ ID NO: 84 - protein, 12 aa, unknown
  Bactenecin 1 Cow
RLCRIVVIRVCR

SEQ ID NO: 85 - protein, 21 aa, unknown
  Thanatin Insect
GSKKPVPIIYCNRRTGKCQRM

SEQ ID NO: 86 - protein, 19 aa, unknown
  Brevinin 1T Rana frogs
VNPIILGVLPKVCLITKKC

SEQ ID NO: 87 - protein, 26 aa, unknown
  Ranateurin 1 Rana frog
SMLSVLKNLGKVGLGFVACKINIKQC

SEQ ID NO: 88 - protein, 46 aa, unknown
  Esculentin 1 Rana frogs
GIFSKLGRKKIKNLLISGLKNVGKEVGMDVVRTGIKIAGCKIKGEC

SEQ ID NO: 89 - protein, 17 aa, Limulus polyphemus
RWCFRVCYRGICYRKCR

SEQ ID NO: 90 - protein, 25 aa, unknown
  Androctonin Scorpion
RSVCRQIKICRRRGGCYYKCTNRPY

SEQ ID NO: 91 - protein, 30 aa, Homo sapiens
DCYCRIPACIAGERRYGTCIYQGRLWAFCC

SEQ ID NO: 92 - protein, 38 aa, unknown
  beta-defensin cow
NPVSCVRNKGICVPIRCPGSMKQIGTCVGRAVKCCRKK

SEQ ID NO: 93 - protein, 18 aa, unknown
  theta-defensin monkey
GFCRCLCRRGVCRCICTR

SEQ ID NO: 94 - protein, 40 aa, unknown
  defensin (sapecin A) insect
ATCDLLSGTGINHSACAAHCLLRGNRGGYCNGKAVCVCRN

SEQ ID NO: 95 - protein, 46 aa, unknown
  Thionin (crambin) plant
TTCCPSIVARSNFNVCRIPGTPEAICATYTGCIIIPGATCPGDYAN

SEQ ID NO: 96 - protein, 50 aa, unknown
  defensin from radish
QKLCQRPSGTWSGVCGNNNACKNQCIRLEKARHGSCNYVFPAHCICYFPC

SEQ ID NO: 97 - protein, 44 aa, Drosophila melanogaster
DCLSGRYKGPCAVWDNETCRRVCKEEGRSSGHCSPSLKCWCEGC

SEQ ID NO: 98 - protein, 25 aa, Homo sapiens
DTHFPICIFCCGCCHRSKCGMCCKT

SEQ ID NO: 99 - protein, 44 aa, unknown
  Bac 5 Cow
RFRPPIRRPPIRPPFYPPFRPPIRPPIFPPIRPPFRPPLGRPFP

SEQ ID NO: 100 - protein, 39 aa, unknown
  PR-39 Pig
RRRPRPPYLPRPRPPPFFPPRLPPRIPPGFPPRFPPRFP

SEQ ID NO: 101 - protein, 20 aa, unknown
  Pyrrhocoricin Insect
VDKGSYLPRPTPPRPIYNRN

SEQ ID NO: 102 - protein, 24 aa, Homo sapiens
DSHAKRHHGYKRKFHEKHHSHRGY

SEQ ID NO: 103 - protein, 19 aa, Unknown
  ECP19
RPPQFTRAQWFAIQHISLN

SEQ ID NO: 104 - protein, 23 aa, Unknown
  MSI-594
GIGKFLKKAKKGIGAVLKVLTTG

SEQ ID NO: 105 - protein, 35 aa, Unknown
  TL-ColM
METLTVHAPSPSTNLPSYGNGAFSLSAPHVPGAGP

SEQ ID NO: 106 - protein, 18 aa, Unknown
  SBO
KLKKIAQKIKNFFAKLVA

SEQ ID NO: 107 - protein, 26 aa, unknown
  Macedocin
GKNGVFKTISHECHLNTWAFLATCCS

SEQ ID NO: 108 - protein, 22 aa, unknown
  Macedocin (Trunc)
GKNGVFKTISHECHLNTWAFLA

SEQ ID NO: 109 - protein, 28 aa, unknown
  D16
ACKLKSLLKTLSKAKKKKLKTLLKALSK

SEQ ID NO: 110 - protein, 17 aa, unknown
  CPF-C1
GFGSLLGKALRLGANVL

SEQ ID NO: 111 - protein, 34 aa, unknown
  TL-ColM
ETLTVHAPSPSTNLPSYGNGAFSLSAPHVPGAGP

SEQ ID NO: 112 - protein, 26 aa, unknown
  TM-174E
LISKGWPYLLVVVLGATIYFWGNSNG

SEQ ID NO: 113 - protein, 45 aa, unknown
  ECP45
RPPQFTRAQWFAIQHISLNPPRCTIAMRAINNYRWRCKNQNTFLR

SEQ ID NO: 114 - protein, 50 aa, unknown
  ColicinE3_1-51 (S37F)
SGGDGRGHNTGAHSTSGNINGGPTGLGVGGGASDGFGWSSENNPWGGGSG

SEQ ID NO: 115 - protein, 68 aa, unknown
  ColicinE3_1-69 (S37F)
SGGDGRGHNTGAHSTSGNINGGPTGLGVGGGASDGFGWSSENNPWGGGSGSGIHWGGGSGHGNGGGNG

SEQ ID NO: 116 - protein, 52 aa, unknown
  ColicinD_1-53
SDYEGSGPTEGIDYGHSMVVWPSTGLISGGDVKPGGSSGIAPSMPPGWGDYS

SEQ ID NO: 117 - protein, 34 aa, Limulus polyphemus
GFKLKGMARISCLPNGQWSNFPPKCIRECAMVSS

SEQ ID NO: 118 - protein, 18 aa, artificial
  synthetic sequence
GFFIPAVILPSIAFLIVP

SEQ ID NO: 119 - protein, 5 aa, artificial
  synthetic sequence
FFVAP

SEQ ID NO: 120 - protein, 13 aa, unknown
  alpha4-helix of T4 lysozyme
PNRAKRVITTFRT

SEQ ID NO: 121 - protein, 27 aa, artificial
  synthetic sequence
KRWVKRVKRVKRWVKRVVRVVKRWVKR

SEQ ID NO: 122 - protein, 25 aa, Artificial Sequence
  synthetic sequence; MW2
GKPGWLIKVALKFKKLIRRPLKRLA

SEQ ID NO: 123 - protein, 5 aa, Artificial
  Linker sequence
GGGGS

SEQ ID NO: 124 - protein, 6 aa, artificial sequence
  His-Tag (6x)
HHHHHH

SEQ ID NO: 125 - protein, 166 aa, Artificial sequence
  Fusion MSI-78-GGGGS-S394 w/o methionine
GIGKFLKKAKKFGKAFVKILKKGGGGSGSMSFKFGKNSEKQLATVKPELQKVARRALELSPYDFTIVQGIRTVAQSAQNIANGTSFLKDPSKSKHVTGDAIDFAPYINGKIDWKDLEAFWAVKKAFEQAGKELGIKLRFGADWNSSGDYHDEIDRGTYDGGHVELV

SEQ ID NO: 126 - protein, 167 aa, Artificial sequence
  Fusion MSI-78-GGGGS-S394
MGIGKFLKKAKKFGKAFVKILKKGGGGSGSMSFKFGKNSEKQLATVKPELQKVARRALELSPYDFTIVQGIRTVAQSAQNIANGTSFLKDPSKSKHVTGDAIDFAPYINGKIDWKDLEAFWAVKKAFEQAGKELGIKLRFGADWNSSGDYHDEIDRGTYDGGHVELV

SEQ ID NO: 127 - protein, 175 aa, Artificial sequence
  Fusion MSI-78-GGGGS-S394-His6
MGIGKFLKKAKKFGKAFVKILKKGGGGSGSMSFKFGKNSEKQLATVKPELQKVARRALELSPYDFTIVQGIRTVAQSAQNIANGTSFLKDPSKSKHVTGDAIDFAPYINGKIDWKDLEAFWAVKKAFEQAGKELGIKLRFGADWNSSGDYHDEIDRGTYDGGHVELVLEHHHHHH

SEQ ID NO: 128 - protein, 291 aa, Artificial Sequence
  Fusion Cathelicidin-BF-KZ144(C14S, C23S, C50S)      w/o methionine
KFFRKLKKSVKKRAKEFFKKPRVIGVSIPFGSKVLRKGDRGDEVSQLQTLLNLSGYDVGKPDGIFGNNTFNQVVKFQKDNSLDSDGIVGKNTWAELFSKYSPPIPYKTIPMPTANKSRAAATPVMNAVENATGVRSQLLLTFASIESAFDYEIKAKTSSATGWFQFLTGTWKTMIENYGMKYGVLTDPTGALRKDPRISALMGAELIKENMNILRPVLKREPTDTDLYLAHFFGPGAARRFLTTGQNELAATHFPKEAQANPSIFYNKDGSPKTIQEVYNLMDGKVAAHRK

SEQ ID NO: 129 - protein, 292 aa, Artificial Sequence
  Fusion Cathelicidin-BF-KZ144(C14S, C23S, C50S)
MKFFRKLKKSVKKRAKEFFKKPRVIGVSIPFGSKVLRKGDRGDEVSQLQTLLNLSGYDVGKPDGIFGNNTFNQVVKFQKDNSLDSDGIVGKNTWAELFSKYSPPIPYKTIPMPTANKSRAAATPVMNAVENATGVRSQLLLTFASIESAFDYEIKAKTSSATGWFQFLTGTWKTMIENYGMKYGVLTDPTGALRKDPRISALMGAELIKENMNILRPVLKREPTDTDLYLAHFFGPGAARRFLTTGQNELAATHFPKEAQANPSIFYNKDGSPKTIQEVYNLMDGKVAAHRK

SEQ ID NO: 130 - protein, 300 aa, Artificial Sequence
  Fusion Cathelicidin-BF-KZ144(C14S, C23S, C50S)-      His6
MKFFRKLKKSVKKRAKEFFKKPRVIGVSIPFGSKVLRKGDRGDEVSQLQTLLNLSGYDVGKPDGIFGNNTFNQVVKFQKDNSLDSDGIVGKNTWAELFSKYSPPIPYKTIPMPTANKSRAAATPVMNAVENATGVRSQLLLTFASIESAFDYEIKAKTSSATGWFQFLTGTWKTMIENYGMKYGVLTDPTGALRKDPRISALMGAELIKENMNILRPVLKREPTDTDLYLAHFFGPGAARRFLTTGQNELAATHFPKEAQANPSIFYNKDGSPKTIQEVYNLMDGKVAAHRKLEHHHHHH

SEQ ID NO: 131 - protein, 169 aa, Artificial Sequence
  Fusion Cathelicidin-BF-S394 w/o methionine
KFFRKLKKSVKKRAKEFFKKPRVIGVSIPFGSMSFKFGKNSEKQLATVKPELQKVARRALELSPYDFTIVQGIRTVAQSAQNIANGTSFLKDPSKSKHVTGDAIDFAPYINGKIDWKDLEAFWAVKKAFEQAGKELGIKLRFGADWNSSGDYHDEIDRGTYDGGHVELV

SEQ ID NO: 132 - protein, 170 aa, Artificial Sequence
  Fusion Cathelicidin-BF-S394
MKFFRKLKKSVKKRAKEFFKKPRVIGVSIPFGSMSFKFGKNSEKQLATVKPELQKVARRALELSPYDFTIVQGIRTVAQSAQNIANGTSFLKDPSKSKHVTGDAIDFAPYINGKIDWKDLEAFWAVKKAFEQAGKELGIKLRFGADWNSSGDYHDEIDRGTYDGGHVELV

SEQ ID NO: 133 - protein, 178 aa, Artificial Sequence
  Fusion Cathelicidin-BF-S394-His6
MKFFRKLKKSVKKRAKEFFKKPRVIGVSIPFGSMSFKFGKNSEKQLATVKPELQKVARRALELSPYDFTIVQGIRTVAQSAQNIANGTSFLKDPSKSKHVTGDAIDFAPYINGKIDWKDLEAFWAVKKAFEQAGKELGIKLRFGADWNSSGDYHDEIDRGTYDGGHVELVLEHHHHHH

SEQ ID NO: 134 - protein, 288 aa, Artificial Sequence
  Fusion MSI-78--KZ144(C14S, C23S, C50S) w/o      methionine
GIGKFLKKAKKFGKAFVKILKKGGGGSGSKVLRKGDRGDEVSQLQTLLNLSGYDVGKPDGIFGNNTFNQVVKFQKDNSLDSDGIVGKNTWAELFSKYSPPIPYKTIPMPTANKSRAAATPVMNAVENATGVRSQLLLTFASIESAFDYEIKAKTSSATGWFQFLTGTWKTMIENYGMKYGVLTDPTGALRKDPRISALMGAELIKENMNILRPVLKREPTDTDLYLAHFFGPGAARRFLTTGQNELAATHFPKEAQANPSIFYNKDGSPKTIQEVYNLMDGKVAAHRK

SEQ ID NO: 135 - protein, 289 aa, Artificial Sequence
  Fusion MSI-78--KZ144(C14S, C23S, C50S)
MGIGKFLKKAKKFGKAFVKILKKGGGGSGSKVLRKGDRGDEVSQLQTLLNLSGYDVGKPDGIFGNNTFNQVVKFQKDNSLDSDGIVGKNTWAELFSKYSPPIPYKTIPMPTANKSRAAATPVMNAVENATGVRSQLLLTFASIESAFDYEIKAKTSSATGWFQFLTGTWKTMIENYGMKYGVLTDPTGALRKDPRISALMGAELIKENMNILRPVLKREPTDTDLYLAHFFGPGAARRFLTTGQNELAATHFPKEAQANPSIFYNKDGSPKTIQEVYNLMDGKVAAHRK

SEQ ID NO: 136 - protein, 297 aa, Artificial Sequence
  Fusion MSI-78-KZ144(C14S, C23S, C50S)-His6
MGIGKFLKKAKKFGKAFVKILKKGGGGSGSKVLRKGDRGDEVSQLQTLLNLSGYDVGKPDGIFGNNTFNQVVKFQKDNSLDSDGIVGKNTWAELFSKYSPPIPYKTIPMPTANKSRAAATPVMNAVENATGVRSQLLLTFASIESAFDYEIKAKTSSATGWFQFLTGTWKTMIENYGMKYGVLTDPTGALRKDPRISALMGAELIKENMNILRPVLKREPTDTDLYLAHFFGPGAARRFLTTGQNELAATHFPKEAQANPSIFYNKDGSPKTIQEVYNLMDGKVAAHRKLEHHHHHH

SEQ ID NO: 137 - protein, 359 aa, Artificial Sequence
  Fusion Cathelicidin-BF-OBPgp279 w/o methionine
KFFRKLKKSVKKRAKEFFKKPRVIGVSIPFGSKNSEKNASIIMSIQRTLASLSLYGGRIDGLFGEKCRGAIILMLNKVYPNFSTNKLPSNTYEAESVFTFLQTALAGVGLYTITIDGKWGGTSQGAIDALVKSYRQITEAERAGSTLPLGLATVMSKHMSIEQLRAMLPTDRQGYAEVYIDPLNETMDIFEINTPLRIAHFMAQILHETACFKYTEELASGKAYEGRADLGNTRPGDGPLFKGRGLLQITGRLNYVKCQVYLREKLKDPTFDITSSVTCAQQLSESPLLAALASGYFWRFIKPKLNETADKDDIYWVSVYVNGYAKQANPYYPNRDKEPNHMKERVQMLAVTKKALGIV

SEQ ID NO: 138 - protein, 360 aa, Artificial Sequence
  Fusion Cathelicidin-BF-OBPgp279
MKFFRKLKKSVKKRAKEFFKKPRVIGVSIPFGSKNSEKNASIIMSIQRTLASLSLYGGRIDGLFGEKCRGAIILMLNKVYPNFSTNKLPSNTYEAESVFTFLQTALAGVGLYTITIDGKWGGTSQGAIDALVKSYRQITEAERAGSTLPLGLATVMSKHMSIEQLRAMLPTDRQGYAEVYIDPLNETMDIFEINTPLRIAHFMAQILHETACFKYTEELASGKAYEGRADLGNTRPGDGPLFKGRGLLQITGRLNYVKCQVYLREKLKDPTFDITSSVTCAQQLSESPLLAALASGYFWRFIKPKLNETADKDDIYWVSVYVNGYAKQANPYYPNRDKEPNHMKERVQMLAVTKKALGIV

SEQ ID NO: 139 - protein, 368 aa, Artificial Sequence
  Fusion Cathelicidin-BF-OBPgp279-His6
MKFFRKLKKSVKKRAKEFFKKPRVIGVSIPFGSKNSEKNASIIMSIQRTLASLSLYGGRIDGLFGEKCRGAIILMLNKVYPNFSTNKLPSNTYEAESVFTFLQTALAGVGLYTITIDGKWGGTSQGAIDALVKSYRQITEAERAGSTLPLGLATVMSKHMSIEQLRAMLPTDRQGYAEVYIDPLNETMDIFEINTPLRIAHFMAQILHETACFKYTEELASGKAYEGRADLGNTRPGDGPLFKGRGLLQITGRLNYVKCQVYLREKLKDPTFDITSSVTCAQQLSESPLLAALASGYFWRFIKPKLNETADKDDIYWVSVYVNGYAKQANPYYPNRDKEPNHMKERVQMLAVTKKALGIVLEHHHHHH

SEQ ID NO: 140 - protein, 268 aa, Artificial Sequence
  Fusion Cathelicidin-BF-PVP-SE1gp146 w/o      methionine
KFFRKLKKSVKKRAKEFFKKPRVIGVSIPFGSMNAAIAEIQRMLIEGGFSVGKSGADGLYGPATKAALQKCIAQATSGNNKGGTLKLTQAQLDKIFPVGASSGRNAKFLKPLNDLFEKTEINTVNRVAGFLSQIGVESAEFRYVRELGNDAYFDKYDTGPIAERLGNTPQKDGDGAKYKGRGLIQVTGLANYKACGKALGLDLVNHPELLEQPEYAVASAGWYWDTRNINAACDADDIVKITKLVNGGTNHLAERTAYYKKAKSVLTS

SEQ ID NO: 141 - protein, 269 aa, Artificial Sequence
  Fusion Cathelicidin-BF-PVP-SE1gp146
MKFFRKLKKSVKKRAKEFFKKPRVIGVSIPFGSMNAAIAEIQRMLIEGGFSVGKSGADGLYGPATKAALQKCIAQATSGNNKGGTLKLTQAQLDKIFPVGASSGRNAKFLKPLNDLFEKTEINTVNRVAGFLSQIGVESAEFRYVRELGNDAYFDKYDTGPIAERLGNTPQKDGDGAKYKGRGLIQVTGLANYKACGKALGLDLVNHPELLEQPEYAVASAGWYWDTRNINAACDADDIVKITKLVNGGTNHLAERTAYYKKAKSVLTS

SEQ ID NO: 142 - protein, 277 aa, Artificial Sequence
  Fusion Cathelicidin-BF-PVP-SE1gp146-His6
MKFFRKLKKSVKKRAKEFFKKPRVIGVSIPFGSMNAAIAEIQRMLIEGGFSVGKSGADGLYGPATKAALQKCIAQATSGNNKGGTLKLTQAQLDKIFPVGASSGRNAKFLKPLNDLFEKTEINTVNRVAGFLSQIGVESAEFRYVRELGNDAYFDKYDTGPIAERLGNTPQKDGDGAKYKGRGLIQVTGLANYKACGKALGLDLVNHPELLEQPEYAVASAGWYWDTRNINAACDADDIVKITKLVNGGTNHLAERTAYYKKAKSVLTSLEHHHHHH

SEQ ID NO: 143 - protein, 164 aa, Artificial Sequence
  Fusion Lycotoxin1-S394 w/o methionine
IWLTALKFLGKHAAKKLAKQQLSKLGSMSFKFGKNSEKQLATVKPELQKVARRALELSPYDFTIVQGIRTVAQSAQNIANGTSFLKDPSKSKHVTGDAIDFAPYINGKIDWKDLEAFWAVKKAFEQAGKELGIKLRFGADWNSSGDYHDEIDRGTYDGGHVELV

SEQ ID NO: 144 - protein, 165 aa, Artificial Sequence
  Fusion Lycotoxin1-S394
MIWLTALKFLGKHAAKKLAKQQLSKLGSMSFKFGKNSEKQLATVKPELQKVARRALELSPYDFTIVQGIRTVAQSAQNIANGTSFLKDPSKSKHVTGDAIDFAPYINGKIDWKDLEAFWAVKKAFEQAGKELGIKLRFGADWNSSGDYHDEIDRGTYDGGHVELV

SEQ ID NO: 145 - protein, 173 aa, Artificial Sequence
  Fusion Lycotoxin1-S394-His6
MIWLTALKFLGKHAAKKLAKQQLSKLGSMSFKFGKNSEKQLATVKPELQKVARRALELSPYDFTIVQGIRTVAQSAQNIANGTSFLKDPSKSKHVTGDAIDFAPYINGKIDWKDLEAFWAVKKAFEQAGKELGIKLRFGADWNSSGDYHDEIDRGTYDGGHVELVLEHHHHHH

SEQ ID NO: 146 - protein, 364 aa, Artificial Sequence
  Fusion MSI-78-OBPgp279 w/o methionine
GIGKFLKKAKKFGKAFVKILKKGGGGSGSKNSEKNASIIMSIQRTLASLSLYGGRIDGLFGEKCRGAIILMLNKVYPNFSTNKLPSNTYEAESVFTFLQTALAGVGLYTITIDGKWGGTSQGAIDALVKSYRQITEAERAGSTLPLGLATVMSKHMSIEQLRAMLPTDRQGYAEVYIDPLNETMDIFEINTPLRIAHFMAQILHETACFKYTEELASGKAYEGRADLGNTRPGDGPLFKGRGLLQITGRLNYVKCQVYLREKLKDPTFDITSSVTCAQQLSESPLLAALASGYFWRFIKPKLNETADKDDIYWVSVYVNGYAKQANPYYPNRDKEPNHMKERVQMLAVTKKALGIVLEHHHHHH

SEQ ID NO: 147 - protein, 357 aa, Artificial Sequence
  Fusion MSI-78-OBPgp279
MGIGKFLKKAKKFGKAFVKILKKGGGGSGSKNSEKNASIIMSIQRTLASLSLYGGRIDGLFGEKCRGAIILMLNKVYPNFSTNKLPSNTYEAESVFTFLQTALAGVGLYTITIDGKWGGTSQGAIDALVKSYRQITEAERAGSTLPLGLATVMSKHMSIEQLRAMLPTDRQGYAEVYIDPLNETMDIFEINTPLRIAHFMAQILHETACFKYTEELASGKAYEGRADLGNTRPGDGPLFKGRGLLQITGRLNYVKCQVYLREKLKDPTFDITSSVTCAQQLSESPLLAALASGYFWRFIKPKLNETADKDDIYWVSVYVNGYAKQANPYYPNRDKEPNHMKERVQMLAVTKKALGIV

SEQ ID NO: 148 - protein, 365 aa, Artificial Sequence
  Fusion MSI-78-OBPgp279-His6
MGIGKFLKKAKKFGKAFVKILKKGGGGSGSKNSEKNASIIMSIQRTLASLSLYGGRIDGLFGEKCRGAIILMLNKVYPNFSTNKLPSNTYEAESVFTFLQTALAGVGLYTITIDGKWGGTSQGAIDALVKSYRQITEAERAGSTLPLGLATVMSKHMSIEQLRAMLPTDRQGYAEVYIDPLNETMDIFEINTPLRIAHFMAQILHETACFKYTEELASGKAYEGRADLGNTRPGDGPLFKGRGLLQITGRLNYVKCQVYLREKLKDPTFDITSSVTCAQQLSESPLLAALASGYFWRFIKPKLNETADKDDIYWVSVYVNGYAKQANPYYPNRDKEPNHMKERVQMLAVTKKALGIVLEHHHHHH

SEQ ID NO: 149 - DNA, 408 bp, Bacteriophage S394
agcttcaaattcggcaaaaacagcgaaaaacagctggcaaccgttaaaccggaactgcagaaagttgcacgtcgtgcactggaactgagcccgtatgattttaccattgttcagggtattcgtaccgttgcacagagcgcacagaatattgcaaatggcaccagctttctgaaagatccgagcaaaagcaaacatgttaccggtgatgcaattgattttgcaccgtatattaacggcaaaatcgattggaaagatctggaagcattttgggcagtgaaaaaagcatttgaacaggcaggtaaagaactgggtattaaactgcgttttggtgcagattggaatagcagcggtgattatcatgatgaaattgatcgtggcacctatgatggtggtcatgttgaactggtt

SEQ ID NO: 150 - DNA, 66 bp, Artificial sequence
  MSI-78 DNA
ggcattggcaaatttctgaaaaaagcgaaaaaatttggcaaagcgtttgtgaaaattctgaaaaaa

SEQ ID NO: 151 - DNA, 90 bp, Artificial Sequence
  nucleic acid encoding Cathelicidin-BF peptide
aaattttttcgcaaactgaaaaaaagcgtgaaaaaacgcgcgaaagaattttttaaaaaaccgcgcgtgattggcgtgagcattccgttt

SEQ ID NO: 152 - DNA, 777 bp, Artificial Sequence
  mutated KZ144 with C14S, C23S and C50S, without      N-terminal methionine
aaagtattacgcaaaggcgataggggtgatgaggtaagtcaactccagacactcttaaatttaagtggctatgatgttggaaagccagatggtatttttggaaataacacctttaatcaggtagttaaatttcaaaaagataatagtctagatagtgatggtattgtaggtaagaatacttgggctgaattattcagtaaatattctccacctattccttataaaactatccctatgccaactgcaaataaatcacgtgcagctgcaactccagttatgaatgcagtagaaaatgctactggcgttcgtagccagttgctactaacatttgcttctattgaatcagcattcgattacgaaataaaagctaagacttcatcagctactggttggttccaattccttactggaacatggaaaacaatgattgaaaattatggcatgaagtatggcgtacttactgatccaactggggcattacgtaaagatccacgtataagtgctttaatgggtgccgaactaattaaagagaatatgaatattcttcgtcctgtccttaaacgtgaaccaactgatactgatctttatttagctcacttctttgggcctggtgcagcccgtcgtttcctgaccactggccagaatgaattagctgctacccatttcccaaaagaagctcaggcaaacccatctattttttataacaaagatgggtcacctaaaaccattcaagaagtttataacttaatggatggtaaagttgcagcacatagaaaa

SEQ ID NO: 153 - DNA, 531 bp, Artificial Sequence
  Fusion MSI-78-GGGGS-S394-His6
catatgggcattggcaaatttctgaaaaaagcgaaaaaatttggcaaagcgtttgtgaaaattctgaaaaaaggcggcggcggcagcggatccatgagcttcaaattcggcaaaaacagcgaaaaacagctggcaaccgttaaaccggaactgcagaaagttgcacgtcgtgcactggaactgagcccgtatgattttaccattgttcagggtattcgtaccgttgcacagagcgcacagaatattgcaaatggcaccagctttctgaaagatccgagcaaaagcaaacatgttaccggtgatgcaattgattttgcaccgtatattaacggcaaaatcgattggaaagatctggaagcattttgggcagtgaaaaaagcatttgaacaggcaggtaaagaactgggtattaaactgcgttttggtgcagattggaatagcagcggtgattatcatgatgaaattgatcgtggcacctatgatggtggtcatgttgaactggttctcgagcaccaccaccaccaccactga

SEQ ID NO: 154 - DNA, 906 bp, Artificial sequence
  Fusion Cathelicidin-BF-KZ144(C14S, C23S, C50S)-      His6
catatgaaattttttcgcaaactgaaaaaaagcgtgaaaaaacgcgcgaaagaattttttaaaaaaccgcgcgtgattggcgtgagcattccgtttggatccaaagtattacgcaaaggcgataggggtgatgaggtaagtcaactccagacactcttaaatttaagtggctatgatgttggaaagccagatggtatttttggaaataacacctttaatcaggtagttaaatttcaaaaagataatagtctagatagtgatggtattgtaggtaagaatacttgggctgaattattcagtaaatattctccacctattccttataaaactatccctatgccaactgcaaataaatcacgtgcagctgcaactccagttatgaatgcagtagaaaatgctactggcgttcgtagccagttgctactaacatttgcttctattgaatcagcattcgattacgaaataaaagctaagacttcatcagctactggttggttccaattccttactggaacatggaaaacaatgattgaaaattatggcatgaagtatggcgtacttactgatccaactggggcattacgtaaagatccacgtataagtgctttaatgggtgccgaactaattaaagagaatatgaatattcttcgtcctgtccttaaacgtgaaccaactgatactgatctttatttagctcacttctttgggcctggtgcagcccgtcgtttcctgaccactggccagaatgaattagctgctacccatttcccaaaagaagctcaggcaaacccatctattttttataacaaagatgggtcacctaaaaccattcaagaagtttataacttaatggatggtaaagttgcagcacatagaaaactcgagcaccaccaccaccaccactga